(19) United States
(12) Patent Application Publication
(10) Pub. No.: US 2006/0084077 A1
(43) Pub. Date: Apr. 20, 2006

(54) Methods and devices for identifying terrorists/criminal suspects by detecting contact of human skin with rubber protective gear

(75) Inventors: Paul Talalay, Baltimore, MD (US); Lingxiang Ye, Baltimore, MD (US); Scott Louis Wehage, Forest Hill, MD (US); Louis Anthony Wehage, Jarrettsville, MD (US)

Correspondence Address: FOLEY AND LARDNER LLP;SUITE 500, 3000 K STREET NW, WASHINGTON, DC, 20007, US

(73) Assignee: Johns Hopkins University

(21) Appl. No.: 11/077,362

(22) Filed: Mar. 11, 2005

Related U.S. Application Data
(60) Provisional application No. 60/552,370, filed on Mar. 12, 2004.

(51) Int. Cl. C12Q 1/68; C12M 1/34

(52) U.S. Cl. 435/6; 435/287.1

(57) ABSTRACT

The present invention relates to devices and methods for detecting a contaminant on a surface, particularly detecting dithiocarbamates on human skin. The devices and methods are further used to identify a suspect or to conduct forensic analysis of a suspect to determine if they have come into contact with protective gear, and particularly rubber protective gear.

### CROSS-REFERENCE TO RELATED PATENT APPLICATIONS

This application claims the benefit of previously filed Provisional Application Ser. No. 60/552,370 filed Mar. 12, 2004.

### STATEMENT OF GOVERNMENT INTEREST

The invention was made with Government support under contract No. N41756-02-C-4703 by the Department of Defense through Technical Support Working Group. The Government may have certain rights in the invention.

### BACKGROUND OF THE INVENTION

The present invention relates generally to the field of forensic analysis of criminal suspects. More particularly, this invention relates to the use of contaminants to identify a suspect or identify suspected activity of a suspect.

Evidence from criminal activities and criminal suspects has long been analyzed to assist law enforcement officials in their attempts to determine who carried out a particular crime. Fingerprint identification is one of the oldest forms of forensic analysis of a crime scene. Fingerprints are often collected and analyzed in order to identify individuals who were at the scene of the crime. The fingerprints that are gathered can be compared with the prints of known individuals. Large numbers of fingerprints are collected and stored everyday in a wide range of applications including forensics, access control, and driver license registration. These fingerprints are kept on file and used to help law enforcement officials identify suspects. In modern times, computers have made it easy to compare a single fingerprint with a large number of fingerprints in a relatively short period of time.

Another forensic technique that is commonly employed involves the collection and analysis of gunshot residues (“GSR”). Firing a weapon produces combustion of the primer and powder of the cartridge. The residue of the combustion products, or unburned primer or powder components, can be used to detect a fired cartridge. Residue may be found on the skin or clothing of the person who fired the gun, on an entrance wound of a victim, or on other target materials at the scene. The discharge of a firearm, particularly a revolver, can deposit residues even to persons at close proximity. The major primer elements are lead (Pb), barium (Ba), or antimony (Sb). Usually, all three are present. Less common elements include aluminum (Al), sulfur (S), tin (Sn), calcium (Ca), potassium (K), chlorine (Cl), or silicon (Si). A mercury-fulminant based primer may be found in ammunition manufactured in Eastern Europe and used in the Middle East. See Zeichner et al., J. Forensic Sci. 37(6) 1567-73 (1992).

The major methods for detection of primer residues are neutron activation analysis (“NAA”), atomic absorption spectrophotometry (“AAS”), and scanning electron microscopy with energy dispersive analysis (“SEM-EDA”). For these methods, samples must be obtained from the skin surfaces of a victim at the scene. Delay in obtaining residues, movement, or washing of the body prior to autopsy will diminish or destroy gunshot residues. The method of collection for residue is quite simple and easily carried out in the field directly onto the gummed surface of a chuck, or holder, applied to the surface (skin or other material) to be tested. See Tassa et al., J. Forensic Sci. 27(3):671-76 (1982a). The chuck, with the residue on the surface, can be directly prepared for examination in the SEM device.

Gas chromatography has been used to identify gun oils on targets, and was very sensitive, even with stored specimens. See Kijewski et al., Z. Rechtsmed 97(2):111-16 (1986). One such method fires a firearm in a plastic bag. See Mach et al., J. Forensic Sci. 23(3):446-55 (1978). The residue may consist of flakes of smokeless powder, a film, or dust of certain characteristic volatilizable organic constituents, which were originally in the powder or were formed upon firing. These residues are subsequently collected from the bag and analyzed by combined gas chromatography-mass spectrometry.

There are many problems with gunshot residue analysis which requires careful evaluation. False positives may be caused by contamination or transfer of GSR to the body by mishandling, or when the body is heavily contaminated by GSR from previous shooting. False negatives result from washing of the hands (when this area is sampled) or by victim wearing gloves. In addition, a rifle or shotgun may not deposit GSR on hands.

Methods of detecting ignitable liquid residues have also been disclosed where a chemist detects the residue from a suspect's skin after the suspect has been identified. One such method uses solid-phase microextraction (“SPME”). SPME employs a sobent-coated fiber to extract compounds that are subsequently desorbed directly into the injection port of a Gas Chromatograph (“GC”) or a High Pressure Liquid Chromatograph (“HPLC”). See Almirall et al., J. Forensic Sci., 45(2):453-61 (1999). The method requires the suspect to place a hand in a nylon bag with the SPME fiber in the headspace above the hand for a period of several minutes. The vapor compounds that interact with the fiber are then desorbed and detected with GC or HPLC.

A more recent addition to the group of forensic tests is DNA analysis. DNA can be isolated from blood, hair, skin cells, or other genetic evidence left at the scene of a crime. DNA forensic analysis relies on the fact that the configuration of a person's DNA is the same in all cells of that individual. Using these sequences, every person could be identified solely by the sequence of their base pairs. Analyzing an entire sequence, however, would be very time consuming. Instead, forensic scientists focus on certain genetic sequences called “markers.” The small number of sequences of DNA in markers vary among individuals a great deal, but are particular to each person. The arrangement of genetic information of a particular individual who is suspected of committing a crime can be analyzed to get a certain probability of a match with the evidence found at the scene of the crime. This comparison can assist in a determination of guilt or innocence. The same process is also useful in establishing the identity of a homicide victim.

Additional forensic tools are needed to identify and prosecute criminals.

### SUMMARY OF THE INVENTION

One embodiment of the invention relates to a device for collecting a sample from a test surface where the sample comprises a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof, that has a substrate, which can be coated with an adhesive; a housing; and optionally, a cap, wherein the housing supports the substrate and wherein the cap protects the substrate from contamination.

Another embodiment of the invention is a method for collecting a sample from a test surface, where the sample contains a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof that includes providing a device comprising a substrate, which can be coated with an adhesive; a housing; and, optionally, a cap, where the housing supports the substrate and where the cap protects the substrate from contamination; optionally, advancing the substrate to protrude from the housing; exposing the substrate; contacting the substrate with the test surface one or more times; optionally, withdrawing the substrate into the housing; and enclosing the substrate.

A further embodiment of the invention is a method for detecting a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof that includes contacting a sample comprising the contaminant collected from a test surface with a cyclocondensation reactant; allowing the contaminant to react with the cyclocondensation reactant; and detecting a cyclocondensation product with HPLC.

Another embodiment of the invention is an assay for detecting a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof that includes providing a device comprising a substrate, which can be coated with an adhesive; a housing; and, optionally, a cap, where the housing supports the substrate and where the cap protects the substrate from contamination; contacting the substrate with a test surface one or more times to collect a sample comprising the contaminant; optionally, removing the contaminant from the substrate; and detecting the contaminant.

A further embodiment of the invention is a method for identifying a suspect who has come into contact with protective gear where the method includes collecting a sample from a person's skin with a device comprising a substrate, which can be coated with an adhesive; a housing; and, optionally, a cap, where the housing supports the substrate and where the cap protects the substrate from contamination, by contacting the substrate with the skin one or more times; optionally, removing the sample from the substrate; and detecting a contaminant on the sample.

Another embodiment of the invention is a method of forensic analysis for determining if a suspect has contacted protective gear that includes collecting a sample from the suspect's skin with a device comprising a substrate, which can be coated with an adhesive; a housing; and, optionally, a cap, where the housing supports the substrate and where the cap protects the substrate from contamination, by contacting the substrate with the skin one or more times; optionally, removing the sample from the substrate; and detecting a contaminant in the sample.

Further, the invention includes as an embodiment a method of identifying members of a criminal network where the method includes introducing protective gear with a contaminant into the network; collecting a sample comprising the contaminant, from a suspected member's skin with a device comprising a substrate, which can be coated with an adhesive; a housing; and, optionally, a cap, where the housing supports the substrate and where the cap protects the substrate from contamination, by contacting the substrate with the skin one or more times; and detecting the presence or absence of the contaminant.

Another embodiment of the invention is a kit that has a device for collecting a sample from a test surface, comprising a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof comprising a substrate, which can be coated with an adhesive; a housing; and, optionally, a cap, where the housing supports the substrate and where the cap protects the substrate from contamination.

### BRIEF DESCRIPTION OF THE DRAWINGS

FIG. 1 is a representation of one embodiment of the device for collecting a sample from a test surface.

FIG. 2a is a representation of the general cyclocondensation reaction.

FIG. 2b is a representation of the cyclocondensation reaction with dithiocarbamate.

FIG. 3 is a representation of sample dithiocarbamates and thiurams reacting with vicinal dithiols to give a cyclocondensation product.

FIG. 4 is a representation of the kinetics and depth of DTC penetration into the skin of a palm of a hand after wearing purple nitrile gloves.

FIG. 5 is a representation of the kinetics of DTC penetration into the skin of a palm of a hand after wearing purple nitrile gloves.

FIG. 6 is a representation of the persistence of DTC contamination over a period of days.

FIG. 7 is a representation of the persistence of DTC contamination over a period of 48 hours.

FIG. 8a is a representation of the reliability of DTC detection based on 7-day glove-wearing history.

FIG. 8b is a representation of the detection of DTCs on the skin of smoking subjects.

FIG. 8c is a representation of the reliability of DTC detection based on 7-day glove-wearing history.

FIG. 8d is a representation of the reliability of DTC detection based on 7-day glove-wearing history.

FIG. 9 is a representation of the detection of DTC remaining on the skin of a right hand after subjects wore gloves for one hour and washed their hands before testing.

FIG. 10 is a representation of the detection of DTC remaining on the skin after immediate Clorox and detergent hand wash.

FIG. 11 is a representation of the transfer of DTCs from a gloved hand to a face.

FIG. 12 is a representation of the transfer of DTCs from a bare, glove-contaminated hand to a face.

FIG. 13 is a representation of the transfer of DTCs from a bare, washed, glove-contaminated hand to a face.

FIG. 14 is a representation of the transfer of DTCs by a handshake.

FIG. 15 is a representation of the transfer of DTCs from a hand to an inanimate object.

FIG. 16 is a representation of the transfer of DTCs from a hand to an inanimate object in a lab that uses rubber gloves (Lab A) and a lab that does not use rubber gloves (Lab B).

FIG. 17 is a representation of the transfer of DTCs from a hand to an inanimate object in a lab that uses rubber gloves (Lab A) and a lab that does not use rubber gloves (Lab B).

FIG. 18 is a representation of the transfer of DTCs from a hand to an inanimate object in a glove user's home and in a non-glove user's home.

FIG. 19 is a representation of the amount of DTC that can be collected with five discs versus the amount that can be collected with one disc placed in five, ten, and fifteen different locations.

FIG. 20 is a representation of the amount of DTC that can be collected with five discs versus the amount that can be collected with one disc placed in five, ten, and fifteen different locations.

FIG. 21 is a representation of the amount of DTC that can be collected with one disc placed in different locations.

FIG. 22 is a representation of the path by which DTC travels from a glove to a first person's hand to an inanimate object and then to a second person's hand.

FIG. 23 is a representation of the PCR results from a skin sample collected according to the methods disclosed herein.

### DETAILED DESCRIPTION OF THE PREFERRED EMBODIMENTS

Before describing the present invention in detail, it must be understood that this invention is not limited to particularly exemplified compositions, formulations, or process parameters as such may vary. It is also to be understood that the terminology used herein is for the purpose of describing particular embodiments of the invention only and is not intended to be limiting. Further, it must be noted that, as used in the specification and the appended claims, the singular forms “a,” “an,” and “the” include plural references unless the content clearly states otherwise. Thus, for example, reference to “a particle” includes a mixture of two or more such particles, reference to “a pharmaceutical” includes mixtures of two or more such agents, and the like.

The present invention relates generally to the field of forensic analysis of criminal suspects. More particularly, this invention relates to the use of contaminants to identify a suspect or to isolate the activity of a suspect. The present invention further relates to devices and methods for collecting a sample from a test surface and to methods for detecting a contaminant on a test surface. Particularly, the invention relates to a method for detecting dithiocarbamates on human skin. The devices and methods are further used to identify a suspect or to conduct forensic analysis of a suspect to determine if the suspect has come into contact with protective gear, and particularly rubber protective gear. Moreover, these methods can be used to determine the candor of a subject and to identify the members of a criminal network. The present invention further relates to a kit that can be used with the devices and methods that are described in this invention.

Many edible plants that belong to the Cruciferae family as well as several other families contain high concentrations of glucosinolates, which are the precursors of isothiocyanates. Most glucosinolates are converted enzymatically to their cognate isothiocyanates by the coexisting, but normally segregated, plant enzyme, myrosinase and by the flora of the human gastrointestinal tract. Myrosinase is released when food is prepared or chewed. In rats and humans, isothiocyanates are metabolized to dithiocarbamates (“DTC”), which are the products of the addition of mercaptans to isothiocyanates. The blood, serum, plasma, and urine of individuals, who have been exposed to vegetables containing isothiocyanates, have been tested for these DTCs using a cyclocondensation reaction HPLC. See Ye et al., Clinica Chimica Acta 316:43-53 (2002).

DTCs are also widely used in the rubber industry to enhance the vulcanization of natural and synthetic rubber. DTCs act to accelerate vulcanization and to impart elasticity to the rubber products. Exemplary products include rubber and nitrile protective gear, such as gloves, aprons, masks, and boots. As a result of the use of DTC during the manufacture of these products, DTC can be found the rubber and nitrile protective gear. Table 1 illustrates the content of dithiocarbamates and thiurams in rubber products determined by cyclocondensation with 1,2-benzenedithiol.

TABLE 1
Content of Rubber Products

| | Total DTC/T |
|---|---|
| Rubber products | (nmol/g) |
| Latex gloves | |
| Safeskin hypoallergenic LPE Latex Exam glove | 43,200 |
| Fisher Scientific Fisher brand glove | 24,100 |
| Evolution One Powder Free Latex Exam glove | 460 |
| Baxter Multi-Flex Non-sterile Latex Laboratory glove | 42 |
| Synthetic polymer gloves | |
| Safeskin Powder Free Purple Nitrile Exam Glove | 25,700 |
| Safeskin Powder Free Nitrile Exam Glove | 20,000 |
| SmartPractice Nitrile Examination Glove | 21,200 |

Gloves, aprons, masks, and boots often come into contact with a person's skin when putting these items on. Rubber that comes into contact with the skin rapidly transfers DTC to skin leaving a DTC contaminant on the surface and even in the top layers of the skin. This contamination can persist for seven days or more. See Example 2. It can even persist after repeated washing with commercial hand soap or detergent and after deliberate decontamination procedures, such as Clorox bleach wash, are employed. See Example 3. Other contaminants, such as carbamates, thiurams, disulfiram, mercaptobenzothiazole (“MBT”) and its derivatives, thioureas, 1,3-diphenylguanidine (“1,3-DPG”), and isothiocyanates can similarly be used for detecting skin contact with protective gear. Those of skill in the art will know of alternate contaminants that can be used in accordance with this invention. Non-DTC containing protective gear is very rare. This type of gear is specially made of non-elastic, non-latex materials and it can be difficult to perform tasks using these materials. Moreover, this type of gear is generally not resistant to organic materials and is easily punctured. Therefore, it would not be effective for many of the uses that are employed by criminals.

It is commonly known that criminals and terrorists (hereinafter referred to as “criminals”) use protective gear for protection against toxic agents, radiological agents, chemical agents, and biological agents. Moreover, protective gear is used by criminals to disguise their participation in a crime, for example, to avoid leaving fingerprints and to avoid coming into contact with blood or other bodily fluids. During use of the protective gear, DTCs and other chemicals from the gloves transfer to the skin of the user leaving a detectable residue. DTC, however, does not transfer directly from protective gear to inanimate objects. Rather, DTC is only transferred to inanimate objects, such as clothing and luggage, after rubber protective gear has been removed and the user touches the objects. The DTC transfers from skin to the object. DTC can even be transferred to other people by shaking hands or by bringing skin into contact with another person's skin. Further, it can be transferred from an inanimate object, such as a telephone, to a user's hand if the inanimate object has previously been touched by someone whose hand is contaminated. DTC is found not only on the surface of a person's skin, but also in the deeper epidermal layers of the skin.

DTC can be used to identify suspects who (1) have used rubber protective gear to protect themselves from exposure to toxins, radioactive materials, chemicals, and biological materials or (2) have used rubber materials in an attempt to disguise their participation in the crime. For example, during a robbery, a criminal may use rubber gloves to avoid leaving fingerprints. Those skilled in the art will know of other uses. For example, a person entering a restricted area that the person is not authorized to access may use rubber gloves to avoid leaving fingerprints. It is to be understood that the terms “criminal” and “suspect” refer to any manner of suspect, including persons who have come into contact with protective gear, either directly or indirectly, but who have not completed or even intended to commit a criminal act.

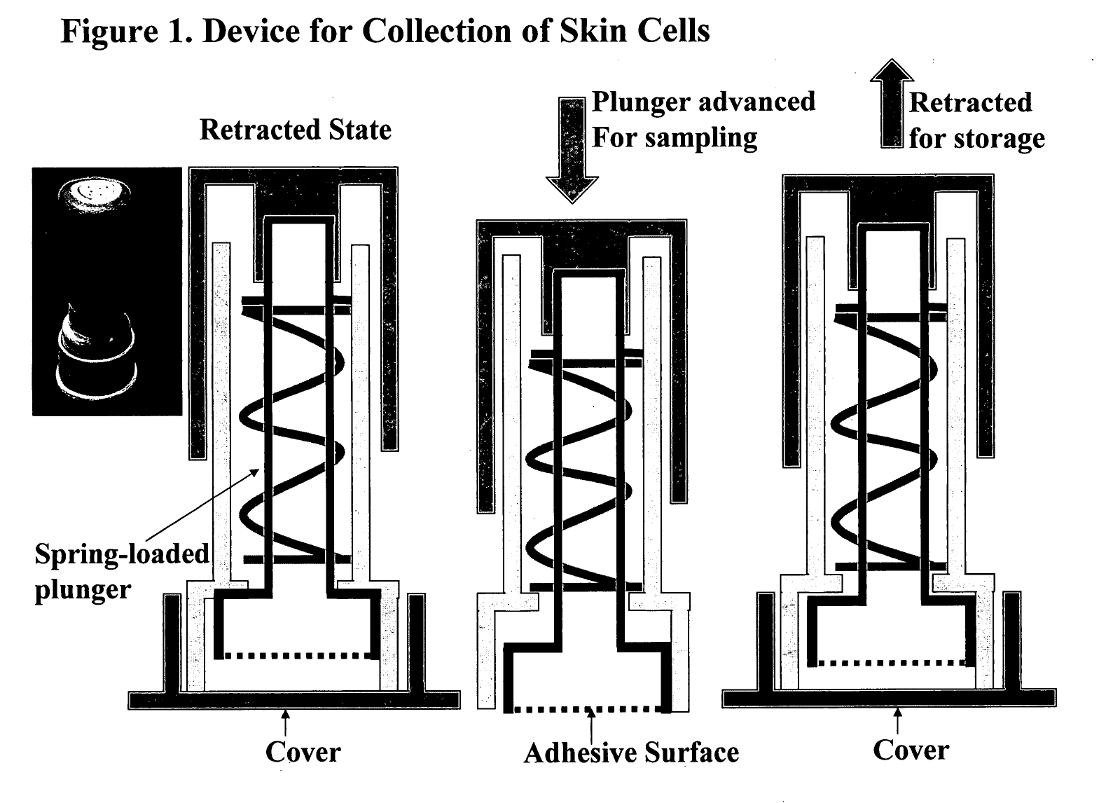

One embodiment of the invention is a device for collecting a sample from a test surface wherein the sample comprises a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof, that has a substrate; a housing; and optionally, a cap, wherein the housing supports the substrate and wherein the cap protects the substrate from contamination. See, e.g., FIG. 1. The substrate can be coated with an adhesive. Alternatively, the substrate can act on its own to collect a sample. For example, the substrate can be sticky to collect a sample on its surface. The test surface can be selected from skin from a human or a mammal, fruit or vegetables, clothing, luggage, or other inanimate object. The sample that is collected can be used to detect the contaminant from the test surface. The contaminant can be from protective gear, such as a rubber glove, a rubber apron, a rubber mask, or a rubber boot.

Skin samples can be taken with a simple, non-invasive mechanical device that houses a substrate, which can be coated with an adhesive. The housing can optionally work in combination with a cap to seal the substrate and protect it, both before and after use, from contamination by the user or by other external sources. The housing can, for example, be made of a polymer, such as delron, polyethylene, and polypropylene. Moreover, in one embodiment, the housing is shaped so as to enclose the substrate on all sides but one, and in another embodiment it can be shaped so as to enclose one side of the substrate. In other words, the housing can surround the substrate or it can merely act as a support to the substrate. Likewise, the cap can be shaped so as to combine with the housing to cover every side of the substrate. In order to seal, the cap is preferably either a snap-on cap or a screw-on cap. The seal can either be airtight or not. If it is not or if no cap is used, it can be sealed completely in some other manner, i.e., with a polymer film, a signed tamper seal, a sealable bag or container, or a tamper-proof pouch. Other shapes and configurations of the housing and cap will be understood by those of skill in the art while maintaining the objective of avoiding contamination by the user or by other external sources.

In a preferred embodiment, the sample is taken from a test surface, such as skin, but it can also be taken from an inanimate object, such as clothing, luggage, corpse, door knob, keyboard, or steering wheel. In a further embodiment, the skin is human skin. Alternatively, the skin can be mammalian skin. In a preferred embodiment, the surface is an external surface. In another preferred embodiment, the sample is not taken from a plant, blood, urine, or internal tissue. Moreover, the sample can contain a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof.

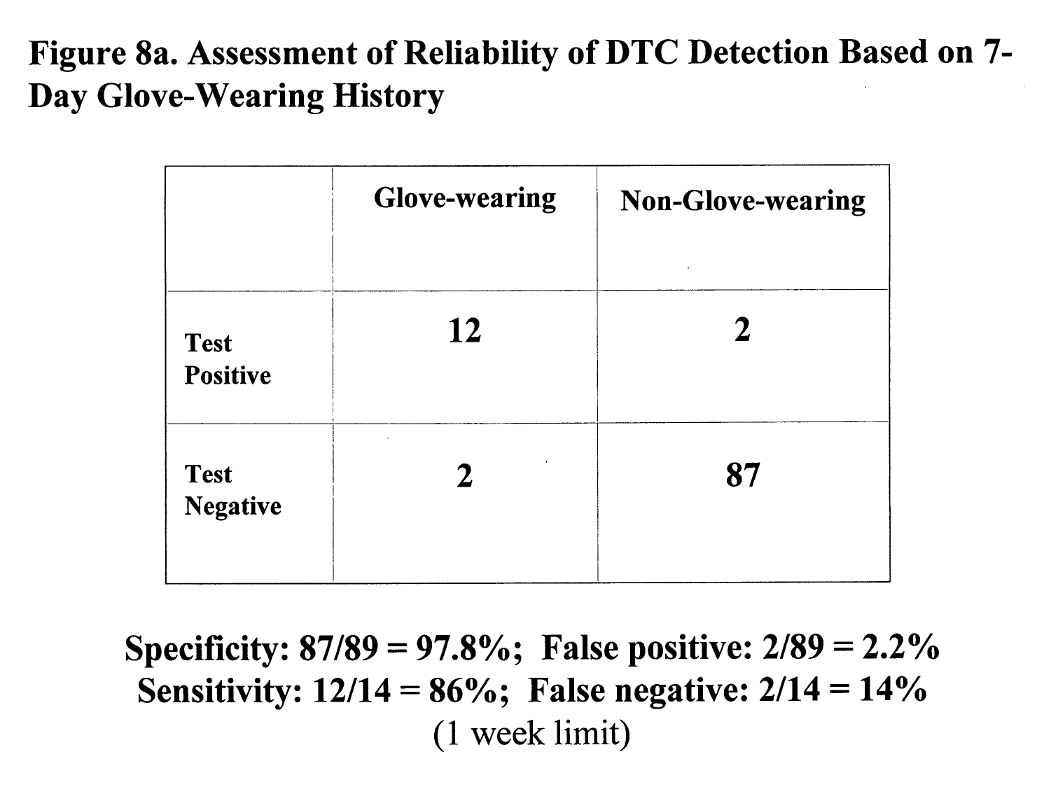

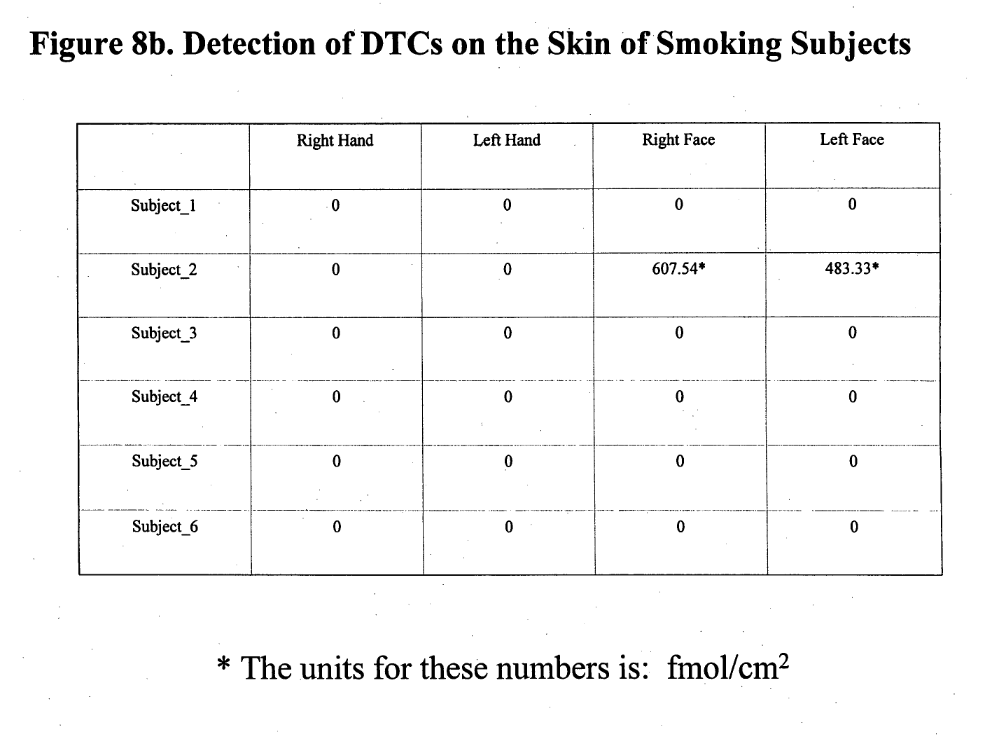

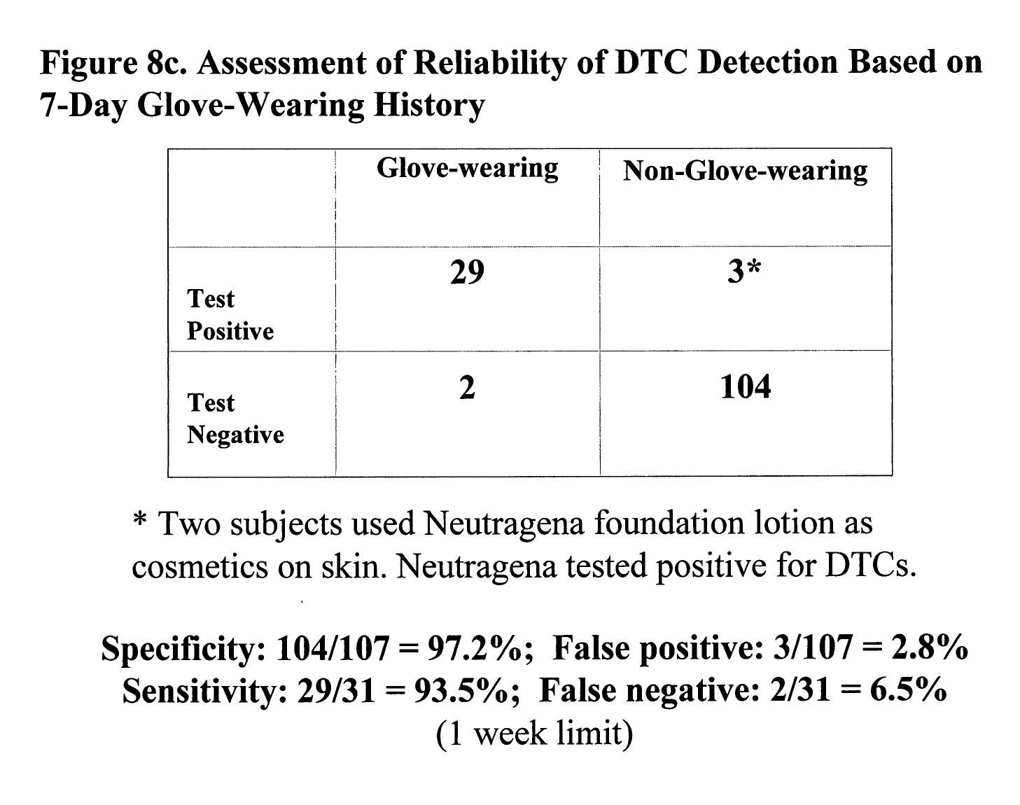

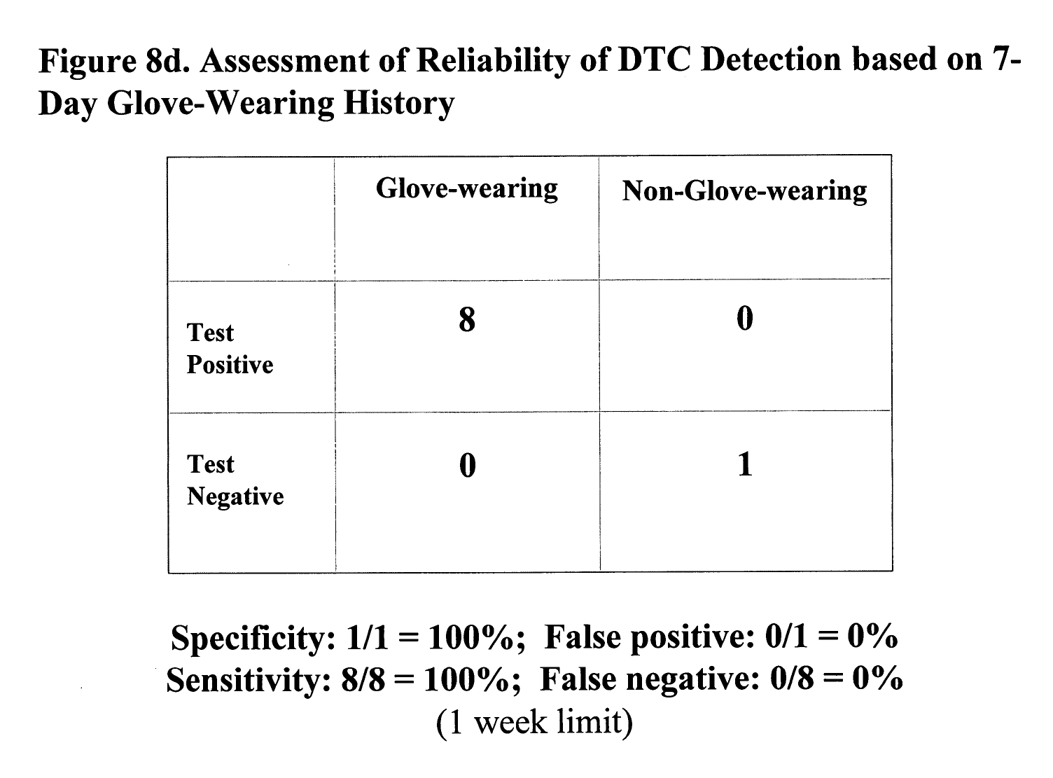

People who eat cruciferous plants do not test positive for DTC on the surface of their skin. Smokers who were tested for contamination and had no known history of contact with rubber materials generally tested negative for contamination. See FIG. 8b. One subject tested positive, but it is unknown whether the individual had previously come into contact with rubber materials. Generally, subjects who have no known history of contact with rubber materials tested negative for contamination. See FIG. 8c. Two subjects, who used Neutragena foundation lotion as cosmetics on their skin, tested positive for contamination. It is unknown whether the individuals had previously come into contact with rubber materials. The Neutragena lotion tested positive for DTCs. Finally, individuals who come into contact with other rubber materials, such as dish gloves, may test positive, however, the levels of DTC contamination are substantially lower than levels found with subjects who have come into contact with rubber protective gear.

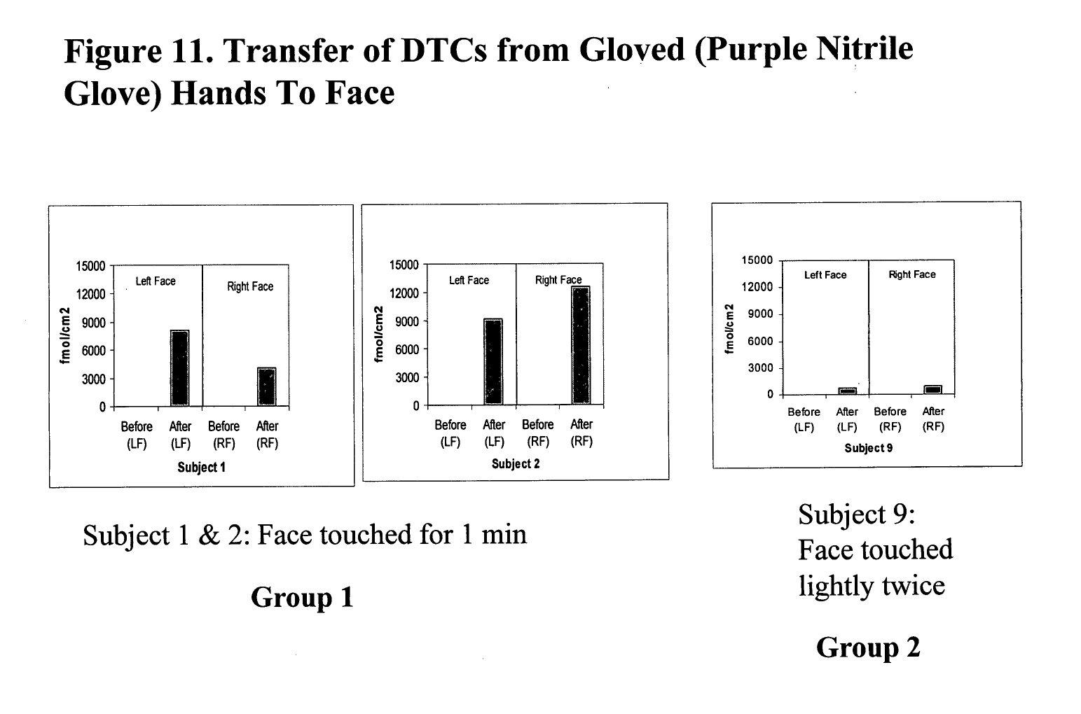

It is important to note that contamination can transfer from a person's hands to a person's face. This can occur by bringing hands into contact with a face while the person is wearing gloves or after a person has taken the gloves off and even after the person has washed their hands. See FIG. 11-13. Two main factors affect the amount of contamination that is detected on a test surface. The first factor is the length of exposure. People who use protective gear for longer periods of time will tend to have higher contamination levels on their hands and face. In addition, people who use protective gear for longer periods of time will tend to have higher contamination levels in their homes. Moreover, people who wear gas masks will have higher levels of contamination on their faces as well. The second factor is how recently the contact occurred.

Further, the sample that is collected can be used to detect the contaminant from the test surface. The sample can also contain skin cells, skin oil, human odor components, DNA, toxic agents, radiological agents, chemical agents, biological agents, or a combination thereof. It can be used to isolate and analyze DNA, to detect the skin cells, skin oil, human odor components, toxic agent, the radiological agent, the chemical agent, the biological agent, or the combination thereof, or to identify an individual by chemical profiling.

The adhesive can, for example, be a polymer. Many commercially available pressure-sensitive adhesives can be used to coat the substrate. Preferred adhesives for use in the construction of the present invention are the homopolymers and interpolymers derived from monomers selected from the C₂ to C₁₀ aliphatic esters of acrylic and methacrylic acid, C₂ to C₁₀ aliphatic vinyl ethers and esters, acrylamides, urethanes and the like. Rubber based adhesives, and other adhesives, containing DTC, carbamates, thiurams, mercaptobenzothiazole (MBT) and its derivatives, thioureas, 1,3-diphenylguanidine (1,3-DPG), or isothiocyanate should be avoided as they may lead to false positive results. A terpolymer of 2-ethylehexyl acrylate, vinyl acetate and tert-butyl acrylamide has been found to be particularly suitable. One such suitable terpolymer has the foregoing monomeric components present in approximate ratios of 60:25:15. It can cover one side or portion of a side or both sides or portion. It can be attached to housing by the same or different adhesive or by any other attaching mechanism known by those skilled in the art. The adhesive can cover the entire substrate or it can cover a portion of the substrate. Preferably, the adhesive covers a relatively flat portion of the substrate that can be placed against the surface that is being tested.

Further, instead of a cap, a protective film can be used to protect the adhesive before and/or after use. Alternatively, a sterile bag, container, or pouch can be used to protect the adhesive before and/or after use. A cap, film, sterile bag, container, and pouch can be used in any combination with each other. In a preferred embodiment, in order to provide protection for the adhesive, a removable protective film can be disposed over the adhesive layer for preventing unwanted debris from adhering to adhesive prior to skin surface sampling. A removable protective film includes a tab to assist in removing film from adhesive prior to skin sampling. Protective film may comprise, for example, a thin film of paper, plastic or other material which is easily severable from the adhesive without removing the adhesive from the substrate when the film is removed. The bag, container, or pouch can be made of a polymer and should not interfere with the sample or contaminate the sample.

In one embodiment, the substrate is made of a polymer. The polymer can be any number of polymeric materials, but preferably it is polyester. The substrate can be any color or clear and it can be any shape that and can take a sample from a surface. Preferably, the substrate is a disc. More preferably, the disc is circular, 22-mm in diameter, and has an area of approximately 19.0 cm². Circular D-Squame polyester discs that are 22-mm in diameter can be obtained from CuDerm Corporation, Dallas Tex. These discs have been used by dermatologists to obtain epidermal cells. Dermatologists use forceps to apply the discs to the skin of hands, sides of the face, or other body parts momentarily and again use forceps to remove the discs.

In a preferred embodiment, the housing has a screw mechanism that turns in order to allow the substrate and/or adhesive to protrude beyond the surface of the housing in order to be able to contact the testing surface without interference. An example mechanism can be found in a glue stick. Alternatively, the housing can include a stamp mechanism, such that when the housing is pressed against the testing surface, the substrate and adhesive are pushed forward to protrude beyond the surface of the housing in order to be able to contact the testing surface without interference. Sample devices, such as Stamp-X, can be obtained from Shachihata Co., Japan, and are distributed by X-Stamper, USA. The Stamp-X device accepts the 22-mm CuDerm disc and has a spring-loaded stamp mechanism.

Not only is it important in many instances that the sample be properly sealed to avoid contamination, but it is also important at times that the sample be properly labeled in order to confirm the chain of custody. Sealing is important before sampling to avoid contamination and tampering. Sealing is important after sampling again to avoid contamination and tampering. Labeling is especially important because it is anticipated that the sampling will take place in the field by law enforcement officers and must be sent to a laboratory to be analyzed. In order to be used as evidence during a trial, it is important for the persons who handle the sample be able to verify the chain of custody and verify that the sample has not been tampered with. In a preferred embodiment, a person takes the sample or another person can record right on the device information such as who took the sample, when the sample was taken, the name, date of birth, and age of the person the sample was taken from, or the item from which the sample was taken, and the circumstances under which the sample was taken. In an alternate embodiment, this information is recorded on a form that is kept with the sample. These methods are used to ensure that the laboratory is the first to open the sample. Procedures similar to those used during urine and other drugs tests may also be employed with the disclosed device and methods. Those skilled in the art will know how to secure the chain of custody and eliminate tampering with the samples.

Another embodiment of the invention is a method for collecting a sample from a test surface, where the sample comprises a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof that includes providing a device comprising a substrate, which can be coated with an adhesive; a housing; and, optionally, a cap, where the housing supports the substrate and where the cap protects the substrate from contamination; optionally, advancing the substrate to protrude from the housing; exposing the substrate; contacting the substrate with the test surface one or more times; optionally, withdrawing the substrate into the housing; and enclosing the substrate. The test surface can be selected from the group consisting of skin that can be human skin or mammal skin, clothing, luggage, or other inanimate object. The sample that is collected can be used to detect the contaminant from the test surface. The contaminant can be from protective gear, such as a rubber glove, a rubber apron, a rubber mask, or a rubber boot.

The sample can further comprise skin cells, skin oil, human odor components, DNA, toxic agents, radiological agents, chemical agents, biological agents, or a combination thereof. The sample can also be used to isolate and analyze DNA or to detect the toxic agent, the radiological agent, the chemical agent, the biological agent, or the combination thereof.

The adhesive used in the device can comprise a polymer and the substrate can comprise a polymer. The polymer can be polyester. The substrate can comprise a disc, where the disc is about 22-mm in diameter. The housing can be a polymer such as delron, polyethylene, or polypropylene. The housing can also comprise a screw mechanism or a stamp mechanism that allows the substrate to protrude beyond the surface of the housing and to be withdrawn into the housing. The cap can comprise a snap-on cap or a screw-on cap. The device can further comprise a film that covers the adhesive. The substrate can be exposed by removing the film and is enclosed by placing the device in a sterile and sealable bag or container.

Several different samples can be taken on different substrates and then combined to detect the contaminant. Alternatively, the same disc can be contacted with the skin surface multiple times in the same or different locations to increase the efficacy of the sampling device. The amount of DTC recovered on the discs depends on the number of discs used on the area of skin that is sampled. Multiple sampling with a single disc has been shown to be equivalent or even more efficient than collecting DTC from the skin using an equivalent number of multiple discs. See Example 5. Thus, samples from five discs are approximately equivalent to five samples from the same disc.

The skin samples can be taken anywhere skin comes into contact with rubber. For example, the palm of a hand, the dorsal skin of the hand, the skin on a face, etc. A person's back typically does not test positive for DTCs even if the person has used rubber gloves extensively because the back does not come into contact with the rubber. The sample skin cells can be used to determine whether the person, who gave the sample, has been in contact with rubber protective articles, as long as one week after contact occurred. This is especially useful to confirm contact with rubber when suspects deny that they have been in contact with rubber. If a person gives a history of contact, then the contact can be verified. The skin cells can also be used to determine whether the person has come into contact with another person who has been in contact with rubber protective articles. If a person's hands are contaminated, then the person's face is almost always contaminated as well.

Condoms may also contain contaminants, such as DTC, that can be detected on the surface of the skin. Condoms are often utilized in sexual assaults in order to avoid leaving semen residue and other bodily fluids. Nevertheless, a condom may leave DTC residue on both the victim and the perpetrator that can be detected using the methods described herein.

A further embodiment of the invention is a method for detecting a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof that includes contacting a sample comprising the contaminant collected from a test surface with a cyclocondensation reactant; allowing the contaminant to react with the cyclocondensation reactant; and detecting a cyclocondensation product with HPLC. The sample can be collected as described above. The method can further comprise contacting an adhesive with a test surface one or more times to collect the sample and, optionally, removing the sample from the adhesive. The adhesive can coat a substrate that is supported by a housing and, optionally, the adhesive can be enclosed with a cap.

The test surface can be selected from the group consisting of skin that can be human skin or mammal skin, clothing, luggage, or other inanimate object. The contaminant can be from protective gear, such as a rubber glove, a rubber apron, a rubber mask, or a rubber boot. The sample can be collected in the field. The method can be used to determine the efficiency of decontamination of a surface.

The adhesive used in the device can comprise a polymer and the substrate can comprise a polymer. The polymer can be polyester. The substrate can comprise a disc, where the disc is about 22-mm in diameter. The housing can be a polymer such as delron, polyethylene, or polypropylene. The housing can also comprise a screw mechanism or a stamp mechanism that allows the substrate to protrude beyond the surface of the housing and to be withdrawn into the housing. The cap can comprise a snap-on cap or a screw-on cap. The device can further comprise a film that covers the adhesive. The substrate can be exposed by removing the film and is enclosed by placing the device in a sterile and sealable bag or container.

DTC can be detected and quantified by a highly specific and exceedingly sensitive cyclocondensation reaction. Thus, DTC can be used to identify contact with rubber and can be used as a tool for criminal investigation. These methods are especially valuable when a person denies coming into contact with protective gear yet tests positive for contact with DTC. In one embodiment, a method for sampling a test surface is envisioned where the substrate is exposed and is further contacted with the testing surface one or more times before being sealed in order to avoid contamination. The test surface can be skin, clothing, luggage, or other inanimate object. The sample that is collected can contain contaminant and can be used to detect the contaminant from the test surface. In addition, it can contain skin cells, skin oil, human odor components, DNA, toxic agents, radiological agents, chemical agents, biological agents, or a combination thereof and be used to isolate and analyze DNA or to detect the toxic agent, the radiological agent, the chemical agent, the biological agent, or the combination thereof. The contaminant can come from protective gear, such as a rubber glove, a rubber apron, a rubber mask, or a rubber boot.

The substrate can be exposed by, for example, removing a protective film from the adhesive or uncapping the housing and, optionally, activating the screw mechanism in the housing to protrude the substrate beyond the surface of the housing, or uncapping the housing and, optionally, activating the stamp mechanism in the housing to protrude the substrate beyond the surface of the housing. The substrate can be contacted with the skin surface by pressing the substrate against the skin one or more times. Further, the housing can be sealed by placing the entire device in a sterile and sealable bag or container or by withdrawing the substrate into the housing and returning the cap to a sealed position. Those skilled in the art will know of other ways to expose and seal the substrate.

In another embodiment, a contaminant is detected by mixing a sample that contains the contaminant with a reaction mixture. The contaminant can be a dithiocarbamate, a carbamate, a thiuram, disulfiram, a mercaptobenzothiazole and its derivatives, thioureas, 1,3-diphenylguanidine, isothiocyanate, and a combination thereof and it can come from protective gear, such as a rubber glove, a rubber apron, a rubber mask, or a rubber boot or from contact with another person, who has come into contact with rubber or rubber contaminated surface or with another contaminated individual. The sample can be collected from a test surface by contacting an adhesive with the test surface one or more times or by contacting more than one substrate with the test surface. Optionally, the sample can be removed from the adhesive and then, mixed with the reaction mixture or the adhesive can be placed directly in the reaction mixture. Alternatively, a second substrate, which contains a cyclocondensation reactant, can be placed in contact with the sample substrate in order to produce the cyclocondensation product. The test surface can be skin, clothing, luggage, or other inanimate object. In addition, the sample can contain skin cells, skin oil, human odor components, DNA, toxic agents, radiological agents, chemical agents, biological agents, or a combination thereof and be used to isolate and analyze DNA or to detect skin cells, skin oil, human odor components, the toxic agent, the radiological agent, the chemical agent, the biological agent, or the combination thereof.

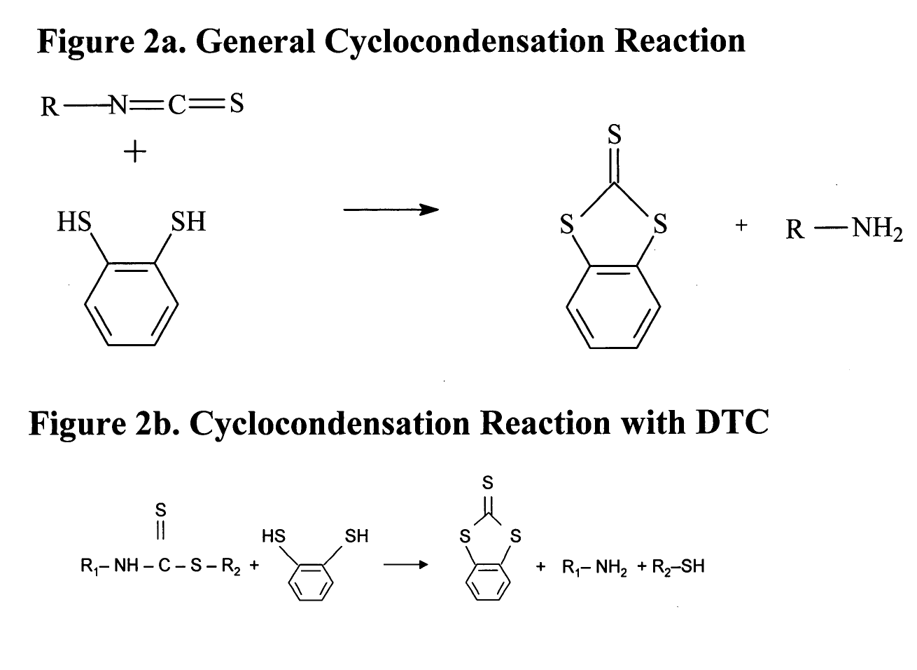

The reaction mixture that is preferably used in this method is made of a cyclocondensation reactant. A general example of a cyclocondensation reaction is shown in FIG. 2a. The highly electrophilic carbon in DTC can undergo successive nucleophilic additions with cyclocondensation reactants containing two sulfhydryl groups on successive carbon atoms. In a preferred embodiment the cyclocondensation reactant is 1,2-benzenedithiol, 3,4-dimercaptotoluene, or 2,3-dimercaptopropanol. Those skilled in the art will know of alternate cyclocondensation reactants that can be used in accordance with this invention. The reaction results in a cyclic thiocarbonyl product and release of the nitrogen atom as an amine. See Zhang et al., Anal. Biochem. 239:160-167 (1996). An assay that utilizes 1,2-benzenedithiol as the vicinal dithiol reagent, for example, results in a reaction product of 1,3-benzodithiole-2-thione. This product can be measured spectroscopically at approximately 365 nm. The reactant, 1,2-benzenedithiol, is commercially available and reacts readily with DTC. The reaction product, 1,3-benzodithiole-2-thione, is very stable under the conditions required for quantitative reaction with DTC and has spectroscopic properties (am of 23,000 M⁻¹cm⁻¹ at 365 nm) suitable for sensitive UV optical detection. Those skilled in the art will know of alternate cyclocondensation products that can be used in accordance with this invention. The reaction is shown below in Formula 1 and in FIG. 2b. See Zhang et al., Anal. Biochem. 239:160-167 (1996).

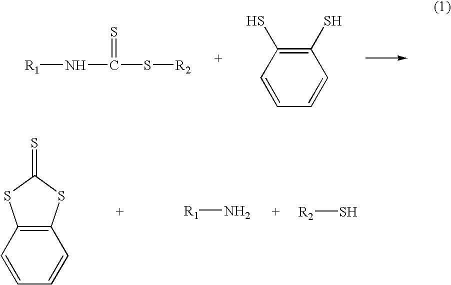

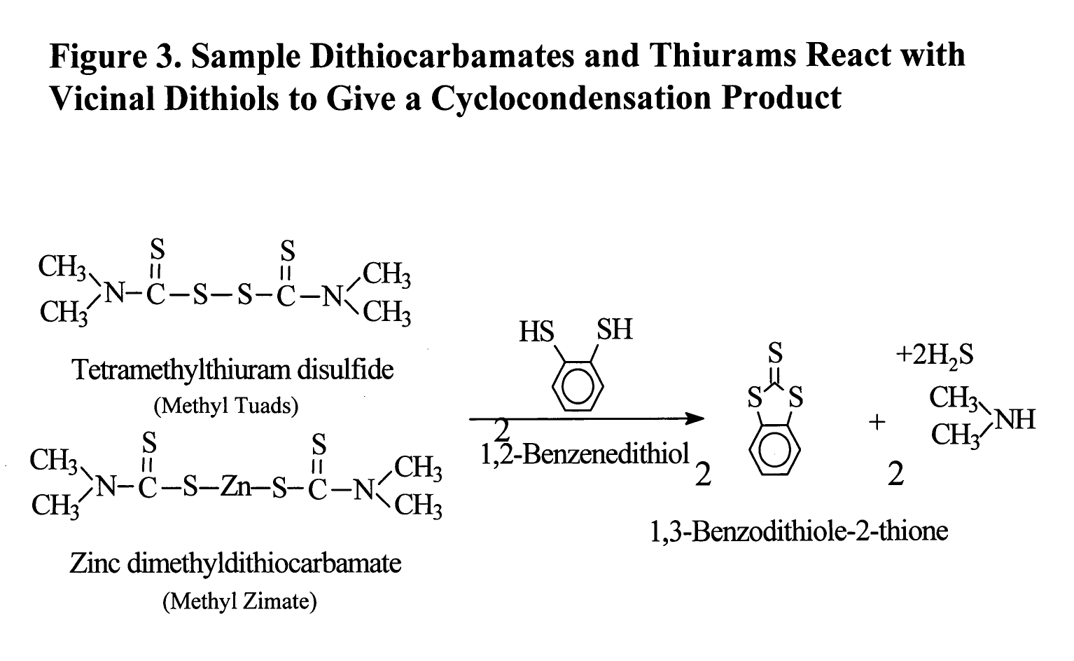

Many dithiocarbamates can participate in the cyclocondensation reaction with 1,2-benzenedithiol, although some compounds react faster than others under the standard assay conditions. Dithiocarbamates comprise diverse chemical types and can be further classified as thiurams, metal-containing DTCs, and N-acetylcysteine mercapturic acids derived from isothiocyanates. Thiurams, such as tetraethylthiuram disulfide (disulfiram), tetraethylthiuram monosulfide, and dipentamethylenethiuram, all react stoichiometrically with 1,2-benzenedithiol under standard reaction conditions. These compounds contain two reaction centers, both of which can condense with 1,2-benzenedithiol. Thus, one equivalent of thiuram in the presence of excess 1,2-benzenedithiol gives rise to two equivalents of 1,3-benzodithiole-2-thione. Metal-containing DTCs are chelated by various metal ions. These compounds also have multiple centers for cyclocondensation. For example, copper dimethyl dithiocarbamates and zinc dimethyl dithiocarbamate forms two equivalents of 1,3-benzodithiole-2-thione and selenium dimethyl dithiocarbamate forms four equivalents of 1,3-benzodithiole-2-thione. Other dithiocarbamates and thiurams include zinc dimethyldithiocarbamate and tetramethylthiuram disulfide. FIG. 3 illustrates the cyclocondensation reaction with these two chemical compounds.

In a preferred embodiment, after the contaminant sample is taken, the adhesive can be placed in a vial equipped with a cap and Teflon/polypropylene septa, suitable for direct automatic sample injection. A reaction mixture is added to the vial. The vials can be flushed with nitrogen and incubated at 65° C. for approximately two hours. After cooling, aliquots of approximately 200 μl of the mixture can be injected manually or automatically onto the chromatographic column. The sensitivity of this method can be increased by reducing the volume of the cyclocondensation reaction mixture or by injecting larger aliquots of the sample onto the column, for example 500 μl. In addition, it can be increased by decreasing the size of the column so the product is diluted in a smaller volume of effluent.

In a preferred embodiment, a Waters 6000A HPLC solvent delivery system equipped with a Model 717 autosampler, a Model 996 photodiode array detector, and a Millennium Chromatography Management system can be used to quantify 1,3-benzodithiole-2-thione. The chromatographic column system consists of a guard column (Li-Christopher 100 RP-18; EM Separations, Gibbstown, N.J.) coupled to an analytical C₁₈ reverse-phase column (Whatman Partisil 10 μm ODS-2; 4.5×250 mm). The column can be operated isocratically with 80% methanol and 20% water at a rate of approximately 2.0 ml/min. The eluates can be monitored at 365 nm by the photodiode array detector and the area of the 1,3-benzodithiole-2-thione peak can be integrated. The 1,3-benzodithiole-2-thione eluate is normally eluted around five to six minutes after it is injected into the system. Between successive sample injections, the column must be washed, preferably for about ten minutes.

The parameters utilized with the HPLC can dramatically effect the sensitivity of detection of the reaction product. In order to increase sensitivity, the reaction products of the cyclocondensation reaction can be subjected to a simple isocratic reverse phase HPLC procedure. This procedure separates 1,3-benzodithiole-2-thione from the impurities in the mixture thereby allowing the HPLC to produce a more accurate measurement. In an isocratic reverse phase HPLC, the solvent composition remains constant throughout the analysis of the elution. In addition, a highly sensitive photodiode array detector can increase sensitivity by more accurately integrating the results of the HPLC. As a result, the HPLC procedure can produce detection levels as low as a few picomoles of DTC with high accuracy and precision.

In a preferred embodiment, a single disc that has been used to sample a skin area by five applications or five discs used to sample the same area are introduced into a 20 ml scintillation vial fitted with a polyethylene cone cap and containing 2.2 ml of a reaction mixture of 25 mmol/l potassium phosphate buffer, pH 8.5, 10 mmol/l 1,2-benzenedithiol and 50% acetonitrile. The mixture is heated for approximately two hours at 65° C., cooled, and diluted with 2.2 ml water. The mixture is then passed over a C₁₈ Sep-Pak Cartridge (50 mg, Waters Part No. WAT054955). The Sep-Pak is washed twice with 1 ml portions of 25% acetonitrile in water and then eluted with 0.3 ml of 100% acetonitrile. The eluate is mixed with 0.3 ml of water. Aliquots of the mixture are then automatically injected onto the reverse phase HPLC column (Partisil 10 ODS-2, 4.6×250 mm, flow rate 2 ml/min, mobile phase 20% water and 80% methanol). The reaction of DTC and 1,2-benzenedithiol results in the formation of 1,3-benzodithiole-2-thione, which can be detected at 365 nm.

Alternatively, the contaminant can be detected directly through the use of a spectrophotometer. This method has the ability to measure the contaminant in parts per billion.

A further embodiment of the invention is an assay for detecting a contaminant. The assay utilizes the device described above, which utilizes a substrate, which can be coated with an adhesive, and a housing; and, optionally includes a cap. The housing supports the substrate and the cap protects the substrate from contamination. As discussed above, the cap can be replaced with a film that protects the substrate from contamination. The assay further comprises contacting the substrate of the device with a test surface one or more times to collect a sample. The adhesive used in the device can comprise a polymer and the substrate can comprise a polymer. The polymer can be polyester. The substrate can comprise a disc, where the disc is about 22-mm in diameter. The housing can be a polymer such as delron, polyethylene, or polypropylene. The housing can also comprise a screw mechanism or a stamp mechanism that allows the substrate to protrude beyond the surface of the housing and to be withdrawn into the housing. The cap can comprise a snap-on cap or a screw-on cap. The device can further comprise a film that covers the adhesive. The substrate can be exposed by removing the film and is enclosed by placing the device in a sterile and sealable bag or container.

The test surface can be skin or an inanimate object, such as clothing, luggage, television, or other common household surfaces and objects. The sample can contain a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof. In addition, it can contain skin cells, skin oil, DNA, toxic agents, radiological agents, chemical agents, biological agents, or a combination thereof and be used to isolate and analyze DNA or to detect skin cells, skin oil, human odor components, the toxic agent, the radiological agent, the chemical agent, the biological agent, or the combination thereof. The contaminant can come from protective gear, such as a rubber glove, a rubber apron, a rubber mask, or a rubber boot.

Once the sample is on the surface of the substrate, the sample can either be removed from the substrate and then brought into contact with the cyclocondensation reactant or it can be brought into contact with the cyclocondensation reactant while it is still in contact with the adhesive. Alternatively, a second substrate, which contains a cyclocondensation reactant, can be placed in contact with the sample substrate in order to produce the cyclocondensation product. Finally, the cyclocondensation product is detected and measured with HPLC or some other method. It is important to note that if a surface that is being tested lacks contaminants, the procedure can be followed with the same steps, but the level of detection will be approximately zero. The cyclocondensation reactant can be selected from the group consisting of 1,2-benzenedithiol, 3,4-dimercaptotoluene, and 2,3-dimercaptopropanol. Those skilled in the art will know of other suitable dithiol reagents that are characterized by the presence of two thiol groups on adjacent or nearby carbon atoms. The cyclocondensation product can be 1,3-benzodithiole-2-thione. Those skilled in the art will know that alternate reagents will lead to different cyclocondensation products, which have known or identifiable characteristics.

Another embodiment of this invention is a method for identifying a suspect who has come into contact with protective gear. Often times, law enforcement officials do not know who committed a crime or an act of terrorism. In these instance, the method includes collecting a sample from a person's skin with a device comprising a substrate, which can be coated with an adhesive; a housing; and, optionally, a cap, where the housing supports the substrate and where the cap protects the substrate from contamination. The sample can be collected by touching the skin surface one or more times and it can contain a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof. The contaminant can be from protective gear, such as a rubber glove, a rubber apron, a rubber mask, or a rubber boot. The sample can then be contacted with a cyclocondensation reactant to produce a cyclocondensation product. Finally, the presence or absence of the cyclocondensation product can be detected with HPLC. Alternatively, the contaminant can be detected directly through the use of a spectrophotometer. If the person's skin tests positive for the cyclocondensation product, then the person can be added to the list of suspects. If the person's skin tests negative for the cyclocondensation product, then alternate testing may be required to determine whether the person should be a suspect. In addition, the sample can contain skin cells, skin oil, human odor components, DNA, toxic agents, radiological agents, chemical agents, biological agents, or a combination thereof and be used to isolate and analyze DNA, to detect skin cells, skin oil, human odor components, the toxic agent, the radiological agent, the chemical agent, the biological agent, or the combination thereof, or to identify an individual by chemical profiling. The contaminant can come from protective gear, such as a rubber glove, a rubber apron, a rubber mask, or a rubber boot. The suspect can be a criminal or a terrorist. The sample can be collected in the field. The method can also be used to determine the efficiency of decontamination of a surface.

The adhesive used in the device can comprise a polymer and the substrate can comprise a polymer. The polymer can be polyester. The substrate can comprise a disc, where the disc is about 22-mm in diameter. The housing can be a polymer such as delron, polyethylene, or polypropylene. The housing can also comprise a screw mechanism or a stamp mechanism that allows the substrate to protrude beyond the surface of the housing and to be withdrawn into the housing. The cap can comprise a snap-on cap or a screw-on cap. The device can further comprise a film that covers the adhesive. The substrate can be exposed by removing the film and is enclosed by placing the device in a sterile and sealable bag or container. The cyclocondensation reactant can be selected from the group consisting of 1,2-benzenedithiol, 3,4-dimercaptotoluene, and 2,3-dimercaptopropanol. The cyclocondensation product can be 1,3-benzodithiole-2-thione.

In a further embodiment, the invention presents a method of forensic analysis for determining if a suspect has contacted protective gear. Once a suspect has been identified, it is often helpful to determine whether the person has come into contact with protective gear. This method is particularly useful if the person has denied coming into contact with protective gear. In these instance, the method includes collecting a sample from the suspect's skin with a device comprising a substrate, which can be coated with an adhesive; a housing; and, optionally, a cap, where the housing supports the substrate and where the cap protects the substrate from contamination, by contacting the substrate with the skin one or more times. The adhesive used in the device can comprise a polymer and the substrate can comprise a polymer. The polymer can be polyester. The substrate can comprise a disc, where the disc is about 22-mm in diameter. The housing can be a polymer such as delron, polyethylene, or polypropylene. The housing can also comprise a screw mechanism or a stamp mechanism that allows the substrate to protrude beyond the surface of the housing and to be withdrawn into the housing. The cap can comprise a snap-on cap or a screw-on cap. The device can further comprise a film that covers the adhesive. The substrate can be exposed by removing the film and is enclosed by placing the device in a sterile and sealable bag or container.

The suspect can be a criminal or a terrorist. The sample can contain a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof. The sample can be collected in the field. The method can also be used to determine the efficiency of decontamination of a surface.

The sample can then be contacted with a cyclocondensation reactant to produce a cyclocondensation product. The cyclocondensation reactant can be selected from the group consisting of 1,2-benzenedithiol, 3,4-dimercaptotoluene, and 2,3-dimercaptopropanol and other vicinal dithiols. The cyclocondensation product can be 1,3-benzodithiole-2-thione. Finally, the presence or absence of the cyclocondensation product can be detected with HPLC. Alternatively, the contaminant can be detected directly through the use of a spectrophotometer. If the person's skin tests positive for the cyclocondensation product, then the suspect has come into contact with protective gear. If the person's skin tests negative for the cyclocondensation product, then alternate testing may be required to determine whether the suspect has committed the accused crime. In addition, it can contain skin cells, skin oil, human odor components, DNA, toxic agents, radiological agents, chemical agents, biological agents, or a combination thereof and be used to isolate and analyze DNA or to detect skin cells, skin oil, human odor components, the toxic agent, the radiological agent, the chemical agent, the biological agent, or the combination thereof. The contaminant can come from protective gear, such as a rubber glove, a rubber apron, a rubber mask, or a rubber boot.

Further, the invention includes a method of identifying members of a criminal network that includes introducing protective gear with a contaminant into the network; collecting a sample comprising the contaminant, from a suspected member's skin with a device comprising a substrate, which can be coated with an adhesive; a housing; and, optionally, a cap, where the housing supports the substrate and where the cap protects the substrate from contamination, by contacting the substrate with the skin one or more times; and detecting the presence or absence of the contaminant. It may be useful to identify the members of a criminal network or to track a person's movement through their contact with objects or to track a person's contact with other people, especially those that use protective gear to avoid exposure to hazardous materials or to cover up their participation in a crime. A person, who has come into contact with rubber protective gear, will get a layer of contamination on their skin from the DTC that is in the rubber. That person will then transfer the contamination to any other person that they touch as well as to inanimate objects. In addition, the person will also be able to transfer skin cells, skin oils, and perspiration to objects that they come into contact with.

The sample can comprise a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof. The contaminant can be from protective gear, such as a rubber glove, a rubber apron, a rubber mask, or a rubber boot. The sample can be collected in the field. The method can be used to determine the efficiency of decontamination of a surface.

The sample can further comprise skin cells, skin oil, human odor components, DNA, toxic agents, radiological agents, chemical agents, biological agents, or a combination thereof. The sample can also be used to isolate and analyze DNA or to detect skin cells, skin oil, human odor components, the toxic agent, the radiological agent, the chemical agent, the biological agent, or the combination thereof. The sample can be collected in the field.

The adhesive used in the device can comprise a polymer and the substrate can comprise a polymer. The polymer can be polyester. The substrate can comprise a disc, where the disc is about 22-mm in diameter. The housing can be a polymer such as delron, polyethylene, or polypropylene. The housing can also comprise a screw mechanism or a stamp mechanism that allows the substrate to protrude beyond the surface of the housing and to be withdrawn into the housing. The cap can comprise a snap-on cap or a screw-on cap. The device can further comprise a film that covers the adhesive. The substrate can be exposed by removing the film and is enclosed by placing the device in a sterile and sealable bag or container.

The detection step can include contacting the sample with a cyclocondensation reactant; allowing the contaminant to react with the cyclocondensation reactant; and detecting a cyclocondensation product with HPLC. Alternatively, the contaminant can be detected directly through the use of a spectrophotometer. The cyclocondensation reactant can be selected from the group consisting of 1,2-benzenedithiol, 3,4-dimercaptotoluene, and 2,3-dimercaptopropanol. The cyclocondensation product can be 1,3-benzodithiole-2-thione. The detection step can also be detecting the contaminant with a spectrophotometer.

Another embodiment of the invention is a kit that has a device for collecting a sample from a test surface, comprising a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof comprising a substrate, which can be coated with an adhesive; a housing; and, optionally, a cap, where the housing supports the substrate and where the cap protects the substrate from contamination.

The sample that is collected can be used to detect the contaminant from the test surface. The contaminant can be from protective gear, such as a rubber glove, a rubber apron, a rubber mask, or a rubber boot. The sample can further comprise skin cells, skin oil, human odor components, DNA, toxic agents, radiological agents, chemical agents, biological agents, or a combination thereof. The sample can also be used to isolate and analyze DNA or to detect skin cells, skin oil, human odor components, the toxic agent, the radiological agent, the chemical agent, the biological agent, or the combination thereof. In fact, the sample can be employed to identify an individual's overall chemical profile.

The adhesive used in the device can comprise a polymer and the substrate can comprise a polymer. The polymer can be polyester. The substrate can comprise a disc, where the disc is about 22-mm in diameter. The housing can be a polymer such as delron, polyethylene, or polypropylene. The housing can also comprise a screw mechanism or a stamp mechanism that allows the substrate to protrude beyond the surface of the housing and to be withdrawn into the housing. The cap can comprise a snap-on cap or a screw-on cap. The device can further comprise a film that covers the adhesive. It is also desirable to make this kit portable so it can be taken into the field and a test can be run and analyzed right away. In this regard, the kit further comprises a portable HPLC or other portable detection device.

In an alternate embodiment, skin samples can also be used to gather skin cells for DNA and forensic analysis. This is a simple and non-invasive way to collect skin cells that can be used to isolate and analyze DNA. The samples can be taken from a mammal, preferably a human. Those of ordinary skill in the art would know of methods to isolate DNA from skin cell samples that have been removed from a skin surface. See, e.g., Jeffreys, et. al., Letters to Nature 316: 76-79 (1985). Moreover, those of ordinary skill in the art will know of methods to analyze DNA once it has been isolated.

Microscopes can also prove useful for viewing the sample at a magnified scale. In addition, animals, such as dogs, have proven accurate and reliable for detecting human odor components to determine whether a particular person has been at the scene of a crime. One embodiment is to use the methods and devices disclosed to take a sample containing a human odor component and allow a dog to sniff the sample and identify the suspect. Alternatively, a dog may be presented with more than one device and asked to indicate which device, if any, matches a previously identified odor sample. This process can, for example, help law enforcement officers connect a piece of clothing found at a crime scene with a particular individual, who has been sampled according to the methods disclosed herein.

Despite the use of protective gear, people who work with hazardous materials, such as toxins, radioactive agents, chemicals, and biological materials, are often unable to entirely prevent contact with the hazardous materials. Skin and inanimate object samples can also be used to determine whether a person or an item has come into contact with toxins, radioactive materials, chemicals, and biological materials. These samples may be tested according to known procedures, such as HPLC, CT imaging, MRI imaging, optical imaging, GC Spectroscopy, SPECT imaging, PET imaging, mass spectrometry, and ultrasound imaging.

Toxic agents are substances that cause either permanent or reversible injury to the health of a living thing on contact or absorption, typically by interacting with biological macromolecules such as enzymes and receptors. The term is usually reserved for naturally produced substances that kill rapidly in small quantities, such as the bacterial proteins that cause tetanus and botulism. A toxic agent can be of animal, plant or microbial origin. Toxic agents, for example, include peptide toxins, which are significantly cytotoxic when present intracellularly. Examples of toxins include cytotoxins, metabolic disrupters (inhibitors and activators) that disrupt enzymatic activity, and radioactive molecules that kill all cells within a defined radius of the effector portion. Exemplary toxins include diphtheria toxin, cholera toxin, ricin, 0-Shiga-like toxin (SLT-I, SLT-II, SLT-IIᵥ), LT toxin, C3 toxin, Shiga toxin, pertussis toxin, tetanus toxin, Pseudomonas exotoxin, alorin, saponin, modeccin, and gelanin.

Radiological agents, such as radioactive isotopes, are naturally or artificially created isotopes of a chemical element having an unstable nucleus that decays, emitting alpha, beta, or gamma rays until stability is reached. The stable end product is a nonradioactive isotope of another element, i.e., radium-226 decays finally to lead-206. Exemplary radiological agents are ²²⁴Ac, ²²⁵Ac, ¹¹¹Ag, ⁷²As, ⁷⁷As, ²¹¹At, ²¹⁷At, ¹⁹⁸ Au, ¹⁹⁹Au, ²¹¹Bi, ²¹²Bi, ²¹³Bi, ⁷⁵Br, ⁷⁶Br, ⁷⁷Br, ^{80m}Br, ¹¹C, ⁵⁵Co, ⁵⁷Co, ⁵⁸Co, ⁵¹Cr, ⁶²Cu, ⁶⁴Cu, ⁶⁷Cu, ¹⁵²Dy, ¹⁶⁶Dy, ¹⁶⁹Er, ¹⁸F, ⁵²Fe, ⁵⁹Fe, ²⁵⁵Fm, ²²¹Fr, ⁶⁷Ga, ⁶⁸Ga, ¹⁵⁴⁻¹⁵⁸Gd, ¹⁰⁷Hg, ¹⁹⁷Hg, ²⁰³Hg, ¹⁶¹Ho, ¹⁶⁶Ho, ¹²⁰I, ¹²³I, ¹²⁴I, ¹²⁵I, ¹²⁶I, ¹³¹I, ¹³³I, ¹¹⁰In, ¹¹¹In, ^{113m}In, ^{114m}In, ¹⁹⁴Ir, ¹⁹²Ir, ¹⁷⁷Lu, ⁵¹Mn, ^{52m}Mn, ⁹⁹Mo, ¹³N, ¹⁵O, ^{189m}Os, ³²P, ³³P, ²¹¹Pb, ²¹²Pb, ¹⁰⁹Pd, ¹⁴⁹ Pm, ²¹⁵Po, ¹⁴²Pr, ¹⁴³Pr, ¹⁰⁹Pt, ¹⁹⁷Pt, ²²³Ra, ²²⁶Ra, ^{82m}Rb, ¹⁸⁶Re, ¹⁸⁸Re, ¹⁸⁹Re, ¹⁰³Rh, ¹⁰⁵Rh, ²¹⁹Rn, ⁹⁵Ru, ⁹⁷Ru, ^{103m}Ru, ¹⁰⁵Ru, ¹¹⁹Sb, ⁷⁵Se, ¹⁵³Sm, ⁸³Sr, ⁸⁹Sr, ¹⁶¹Tb, ^{94m}Tc, ⁹⁴Tc, ^{99m}Tc, ^{121m}Te, ^{122m}Te, ^{125m}Te, ²⁰¹Tl, ¹⁶⁵Tm, ¹⁶⁷Tm, ¹⁶⁸Tm, ⁸⁶Y, ⁹⁰Y, ¹⁶⁹Yb, ⁸⁹Zr, or other alpha-, beta-, gamma-, or positron-emitters.

Chemical agents cover a wide variety of harmful chemicals. For example, nerve agents (also known as nerve gases, though these chemicals are liquid at room temperature) are a class of phosphorus-containing organic chemicals (organophosphates) that inhibit the acetylcholinesterase enzyme in animals and which are used as insecticides and chemical weapons. Poisoning by a nerve agent leads to contraction of pupils, profuse salivation, convulsions, involuntary urination and defecation, and eventual death by asphyxiation as control is lost over respiratory muscles. Nerve agents can be absorbed through the skin, requiring that those likely to be subjected to such agents wear a full body suit in addition to a gas mask. Examplary nerve agents include Cyclohexyl Sarin (GF), GE, Sarin (GB), Soman (GD), Tabun (GA), VE, VG, V-Gas, VM, and VX.

Other chemical agents include blister agents, such as Distilled Mustard (HD), Lewisite (L), Nitrogen Mustard (HN-2), Phosgene Oxime (CX), Ethyldichloroarsine (ED), Lewisite 1 (L-1), Lewisite 1 (L-2), Lewisite 1 (L-3), Methyldichloroarsine (MD), Mustard/Lewisite (HL), Mustard/T,Nitrogen Mustard (HN-1), Nitrogen Mustard (HN-3), Phenodichloroarsine (PD), and Sesqui Mustard; blood agents, such as Arsine (SA), Cyanogen Chloride (CK), Hydrogen Chloride, and Hydrogen Cyanide (AC); Choking/Lung/Pulmonary Damaging agents, such as Chlorine (CL), Diphosgene (DP), Cyanide, Nitrogen Oxides, Perflurorisobutylene (PHIB), Phosgene (CG), Red Phosphorous (RP), Sulfur Trioxide-Chlorosulfonic Acid (FS), Teflon and Perflurorisobutylene (PHIB), Titanium Tetrachloride (FM), and Zinc Oxide (HC); incapacitating agents, such as Agent 15, BZ, Cannabinoids, Fentanyls, LSD, and Phenothiazines; riot control/tear agents, such as Bromobenzylcyanide (CA), Chloroacetophenone (CN), Chloropicrin (PS), CNB—(CN in Benzene and Carbon Tetrachloride), CNC—(CN in Chloroform), CNS—(CN and Chloropicrin in Chloroform), CR, and CS; and vomiting agents, such as Adamsite (DM), Diphenylchloroarsine (DA), Diphenylcyanoarsine (DC). Other chemical agents include opiods, opiates, and other narcotics or drugs of abuse. Drugs of abuse can include any drug substance that has the potential for being addictive or misused. For example, it can include steroids, alcohol, and illicit drug substances, among others. A drug need not be illegal to be considered a drug of abuse.

Biological agents can be, for example, viruses and bacteria. Exemplary biological agents are Anthrax, Cholera, Plague, Botulism (Botulinum Toxin), Tularemia, Variola (Smallpox), Q Fever, Staphylococcal Enterotoxin B (Toxin), Brucellosis, Venezuelan Equine Encephalitis, Tricothecene, Viral hemorrhagic fevers (filoviruses [e.g., Ebola, Marburg] and arenaviruses [e.g., Lassa, Machupo]), Nipah virus, Tickborne encephalis ne encephalitis]), Melioidosis (Burkholderia pseudomallei), Psittacosis (Chlamydia psittaci), Typhus fever (Rickettsia prowazekii), viral encephalitis (alphaviruses [e.g., venezuelan equine encephalitis, eastern equine encephalitis, western equine encephalitis]), Water safety threats (e.g., Vibrio cholerae, Cryptosporidium parvum), Hantaviruses, Multidrug-resistant tuberculosis, Nipah virus, Tickborne encephalitis viruses, Tickborne hemorrhagic fever viruses, and Yellow fever.

In a further embodiment, the sampling procedure can be used to monitor the efficiency of decontamination. People who come into contact with rubber often attempt to decontaminate their skin by washing their hands with regular hand soap or detergent. Additionally, a specific bleach decontamination procedure, such as Clorox bleach rinsing, may be employed for decontamination purposes. Those of skill in the art will know of alternative decontamination methods. Skin samples can be taken before and after successive attempts to decontaminate a person's skin in order to determine the efficiency of the decontamination procedures and the efficiency of each successive attempt. The procedures used to test the samples that are taken are the same as the methods used to detect a contaminant on a person's skin.

It is widely known that DTCs are used in the fruit industry as an insecticide. Farmers spray fruit and vegetables with DTC. Another embodiment of the present invention therefore is to test the surface of fruits and vegetables for DTC. The methods described in this invention can also be used to nondestructively test fruits and vegetables that are not supposed to contain DTCs.

Alternative structures, functions, and operations are possible within the scope of the invention. Each publication, patent, and reference referred to in this specification is incorporated herein by reference in its entirety as if the reference had individually been incorporated by reference.

Given the disclosure of the present invention, one versed in the art would appreciate that there may be other embodiments and modifications within the scope and spirit of the invention. Accordingly, all modifications attainable by one versed in the art from the present disclosure within the scope and spirit of the present invention are to be included as further embodiments of the present invention. All of the examples, methods and/or compositions disclosed, and claimed herein can be made and executed without undue experimentation in light of the present disclosure. While the methods and compositions of this invention have been described in terms of preferred embodiments, it will be apparent to those skilled in the art that variations may be applied to the methods and/or compositions and in the steps or in the sequence of steps of the methods described herein without departing from the concept, spirit and scope of the invention. It will be apparent to those skilled in the art that compositions with compounds which are structurally and functionally related may be substituted for compositions with the compounds described herein.

The following examples are merely intended to be exemplary and are not intended to limit the scope of the claims.

### EXAMPLES

### Example 1

### Time Course and Depth of Penetration of DTCs

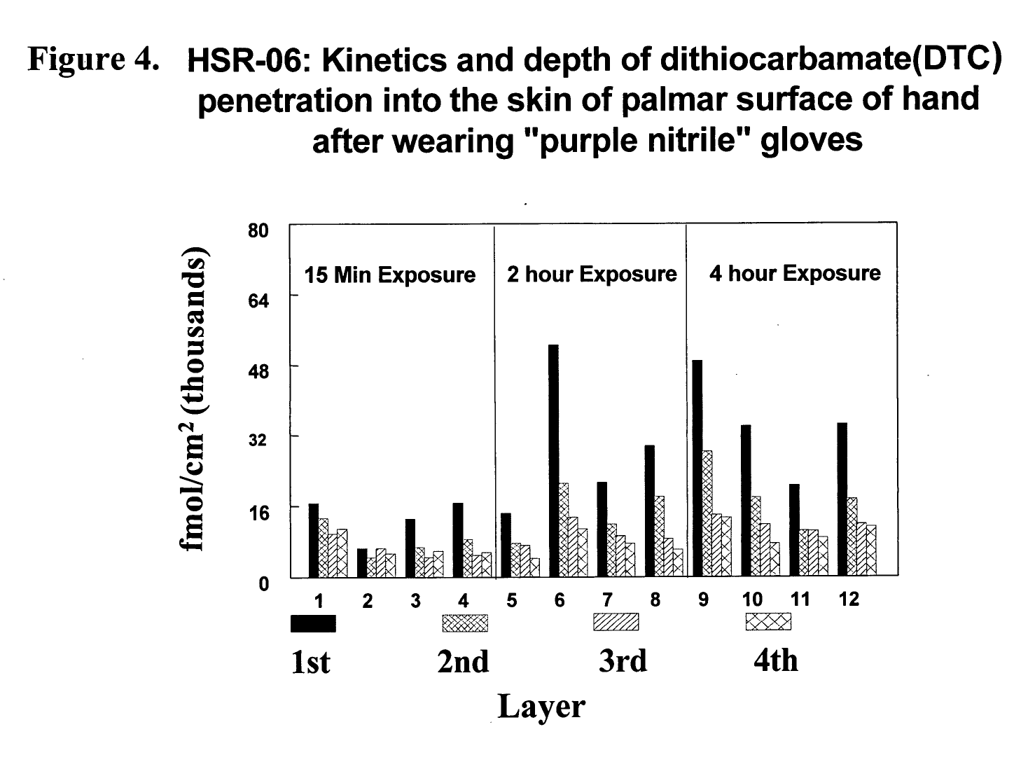

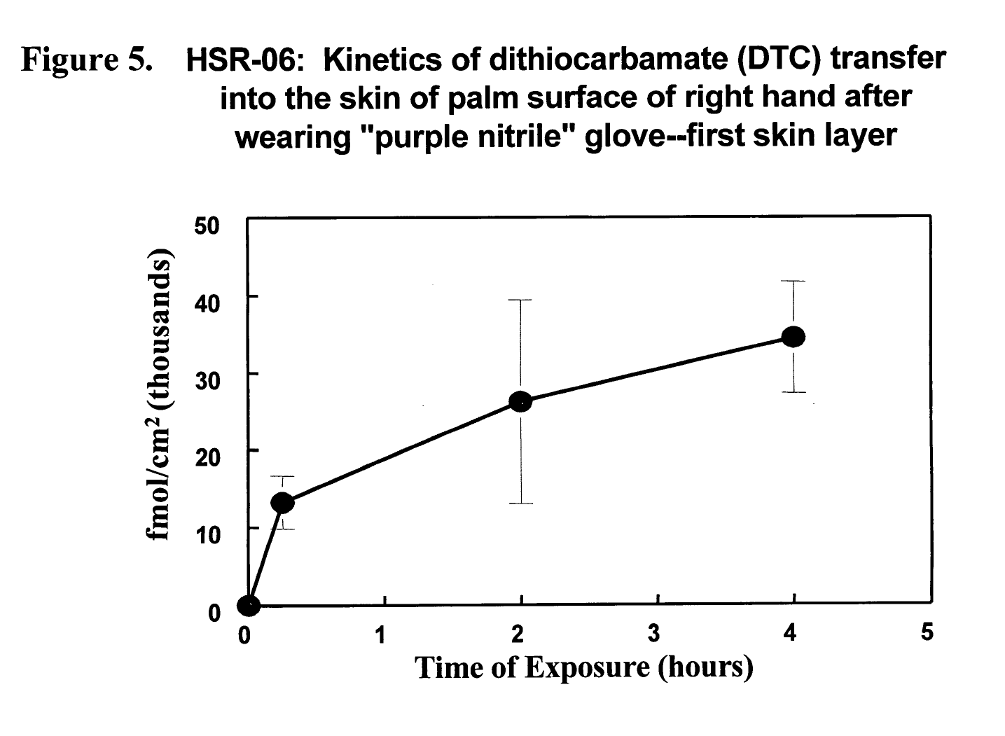

Twelve volunteers who had not worn rubber gloves recently were selected. They were divided into three groups of four subjects. Group 1 wore purple nitrile gloves having a DTC content of 21,700 nmol/g for 15 minutes. Group 2 wore the same purple nitrile gloves for 2 hours. Group 3 wore the same purple nitrile gloves for 4 hours. The transfer and depth of penetration of DTCs was determined by applying CuDerm different discs to the surface of the palm of the right hand four times in rapid succession at the same location. Each sample illustrates the amount of DTC found at a lower level of skin. The discs were assayed by the cyclocondensation method and the results are illustrated in FIG. 4. FIG. 4 shows the results from the four people in Group 1, who were exposed for 15 minutes. The results are expressed as fmol per cm² of sampling area and illustrate how each lower level of skin contains less DTC. The results show that DTC contaminated skin can be detected after just 15 minutes of exposure to rubber gloves and the persistence of DTC is evident even after 4 repeat samples obtained from the same skin area. In addition, FIG. 5 illustrates that the amount of DTC that is transferred from the rubber gloves to the person wearing the gloves and it shows that DTC contamination is detectable after only 15 minutes of exposure. It continues to rise as the length of exposure increases, but starts to level off after about 2 hours of exposure. Relatively brief exposure to rubber gloves is sufficient to produce significant skin contamination, going down at least four layers into the skin.

### Example 2

### Persistence of DTCs After Wearing Purple Nitrile Gloves

In order to determine the persistence of DTCs that have been transferred from gloves to the palm, six subjects who claimed not to have worn gloves recently were chosen for study. Each subject was asked to wear purple nitrile gloves (DTC content of 21,700 nmol/g) on both hands for two hours. The DTCs remaining on the skin surface were measured immediately upon removing the gloves, at two days, four days, and ten days after glove wearing. Five D-Squame adhesive discs (22 mm diameter; area 19 cm²) were applied to different locations on the skin surface of the hand at each time point.

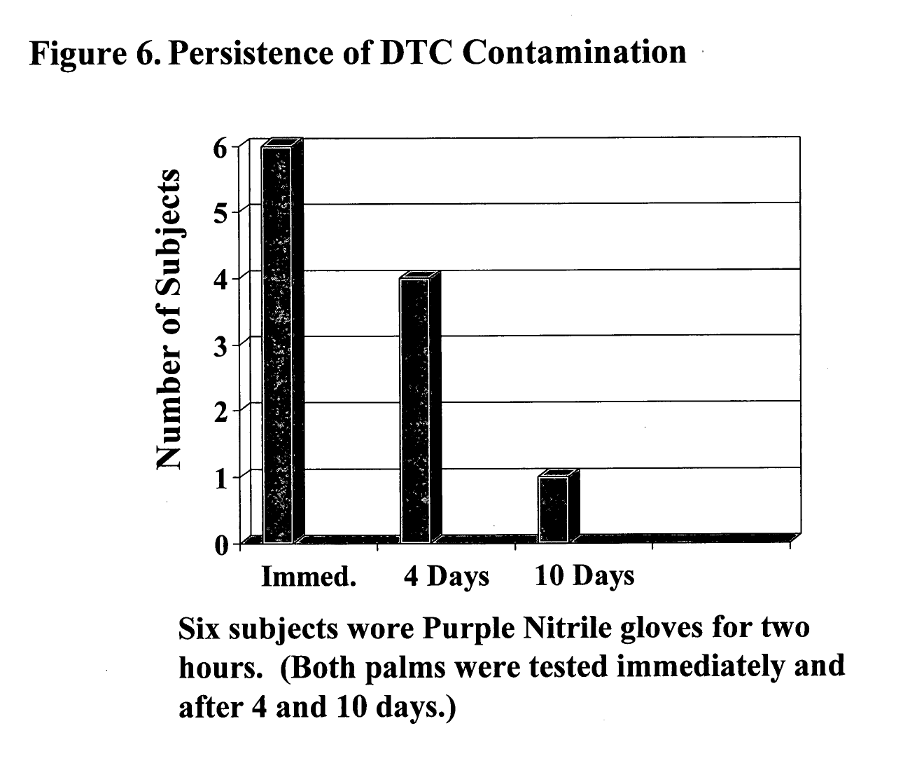

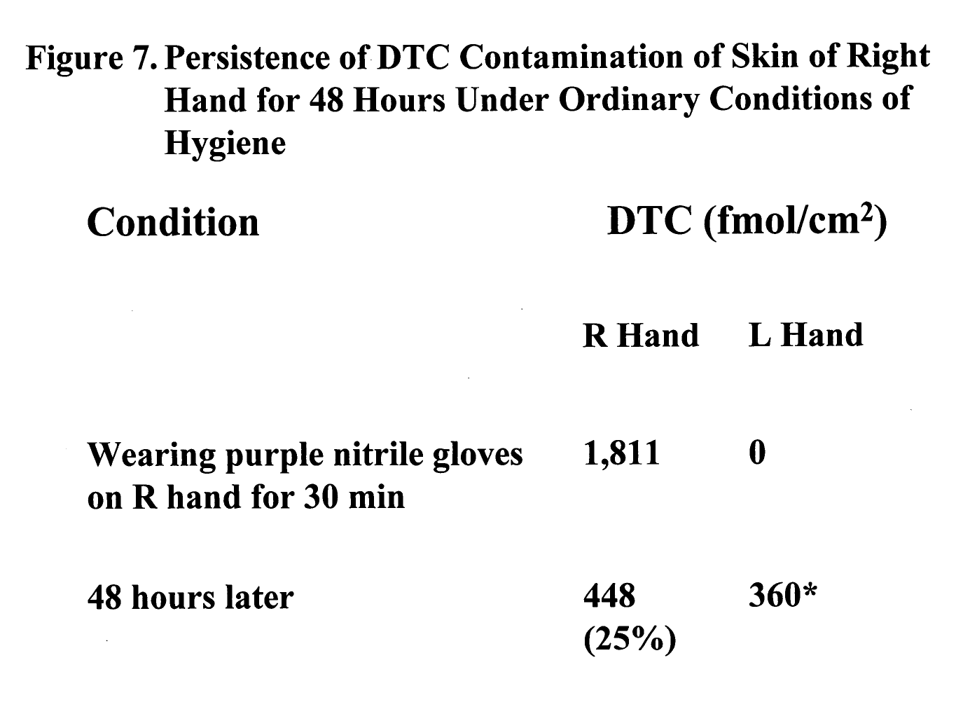

All subjects were negative before wearing the gloves. FIG. 6 illustrates the results of the persistence of the DTC contamination. All subjects tested positive immediately following exposure. Four of the subjects tested remained positive after four days and only one subject remained positive after 10 days. See also FIG. 7. In FIG. 7, after 48 hours, DTC contamination is still evident on the palm of the study volunteer. Interestingly, the contamination was transferred to the volunteer's other (left) hand.

FIG. 8 illustrates the assessment of reliability of DTC detection based on 7-day glove-wearing history. Smokers who were tested for contamination and had no known history of contact with rubber materials generally tested negative for contamination. See FIG. 8b. One subject tested positive, but it is unknown whether the individual had previously come into contact with rubber materials. Generally, subjects who have no known history of contact with rubber materials tested negative for contamination. See FIG. 8c. Two subjects, who used Neutragena foundation lotion as cosmetics on their skin, tested positive for contamination. It is unknown whether the individuals had previously come into contact with rubber materials. The Neutragena lotion tested positive for DTCs.

### Example 3

### Persistence of DTCs After Regular Washing and Clorox Bleach

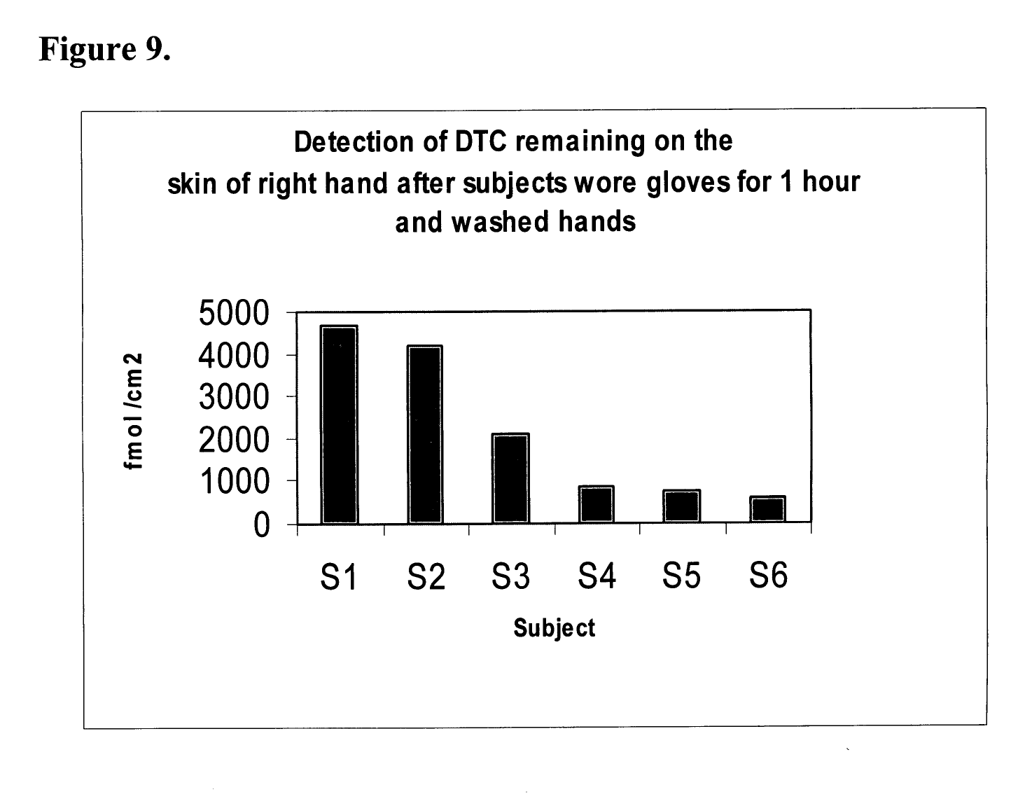

In order to determine the persistence of DTCs that have been transferred from gloves to the palm, six subjects who claimed not to have worn gloves recently were chosen for study. Each subject was asked to wear purple nitrile gloves (DTC content of 21,700 nmol/g) on both hands for one hour. Subjects immediately washed their hands in their normal manner. The DTCs remaining on the skin surface were measured immediately thereafter. Five D-Squame adhesive discs (22 mm diameter; area 19 cm²) were applied to different locations on the skin surface of the hand. The results varied per person and are illustrated in FIG. 9. All subjects exhibited some amount of DTC contamination.

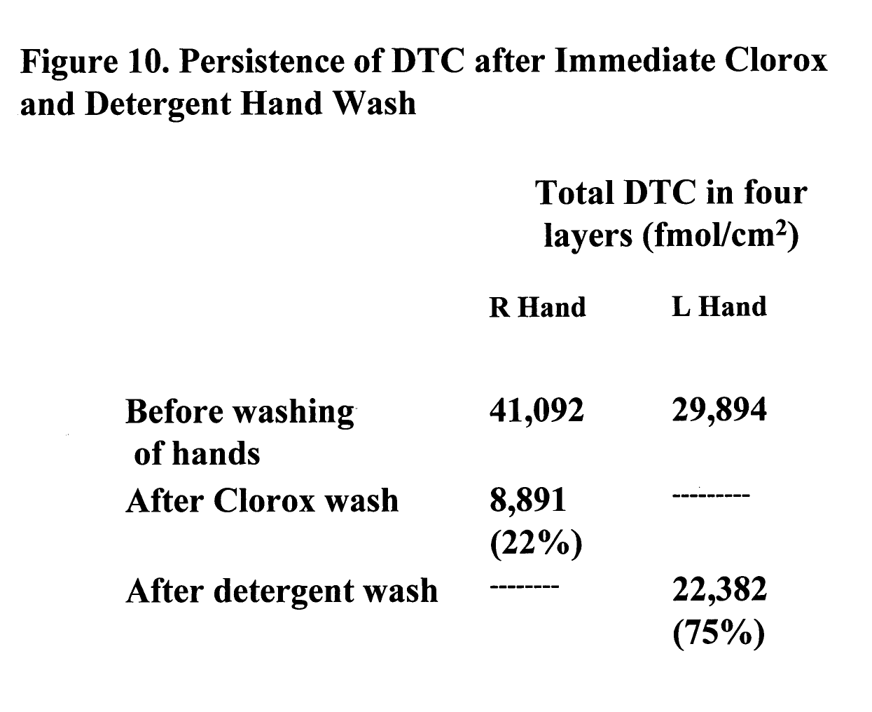

After wearing nitrile gloves on both hands for a period of time, the subject's right hand was immediately washed with Clorox and the subject's left hand was immediately washed with detergent. As illustrated in FIG. 10, the DTC contamination on the right hand decreased to 22% of the original level of DTC contamination. The DTC contamination on the left hand decreased to 75% of the original level of DTC contamination.

### Example 4

### Transfer of DTCs to and from Inanimate Objects and Between Humans

The amount of DTC that is transferred from a gloved hand to a person's face was measured. FIG. 11 illustrates that if a person touches their face for approximately one minute there will be a detectable level of DTC contamination on the person's face. Even if the person only touches their face twice briefly, there will be a detectable level of DTC contamination on the person's face.

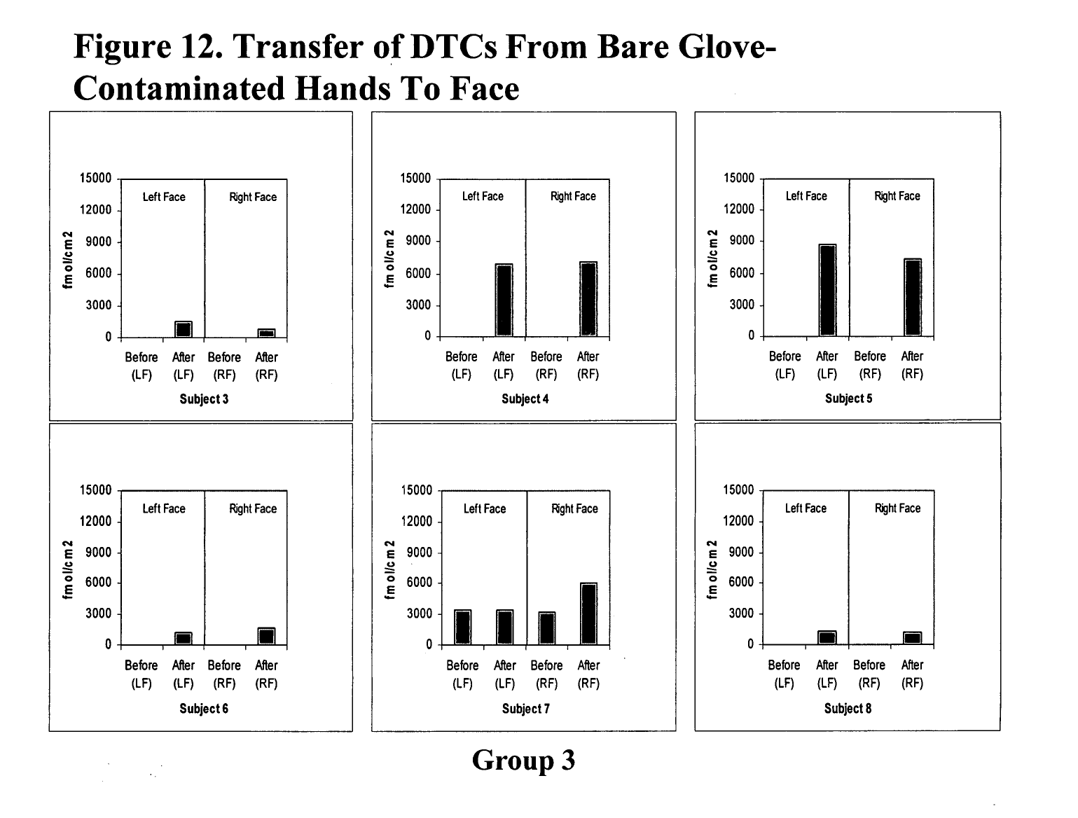

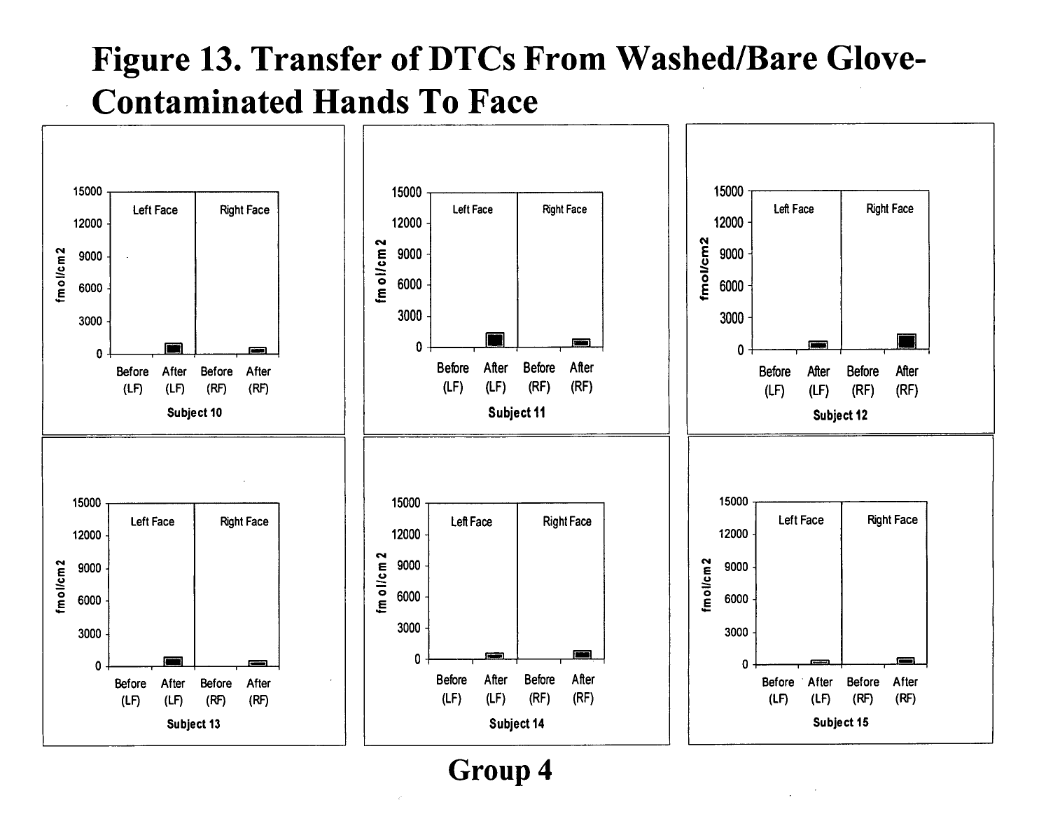

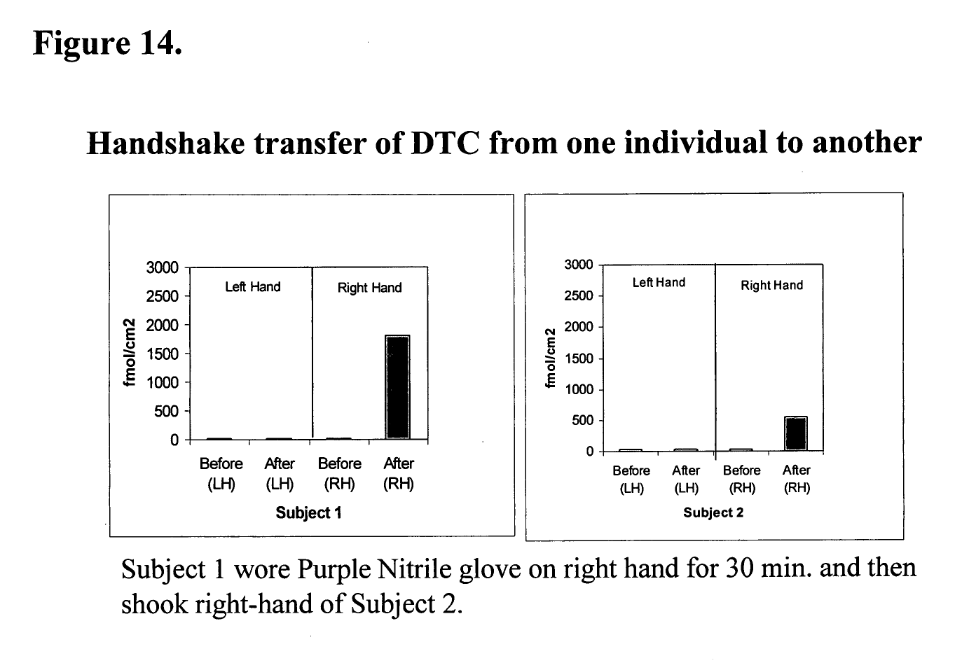

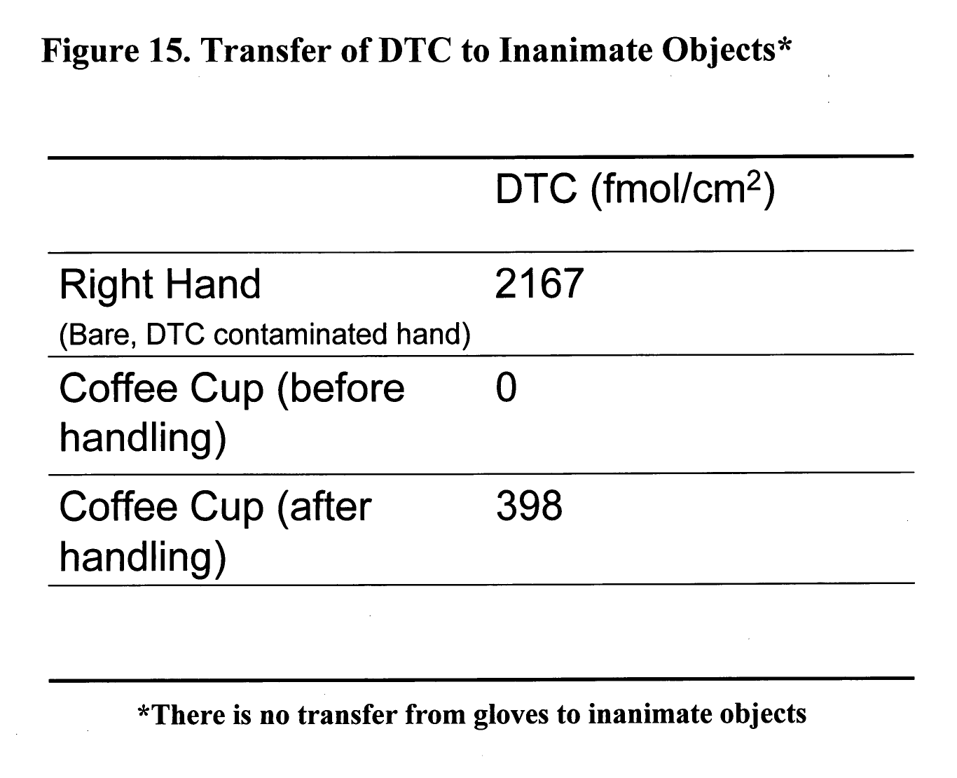

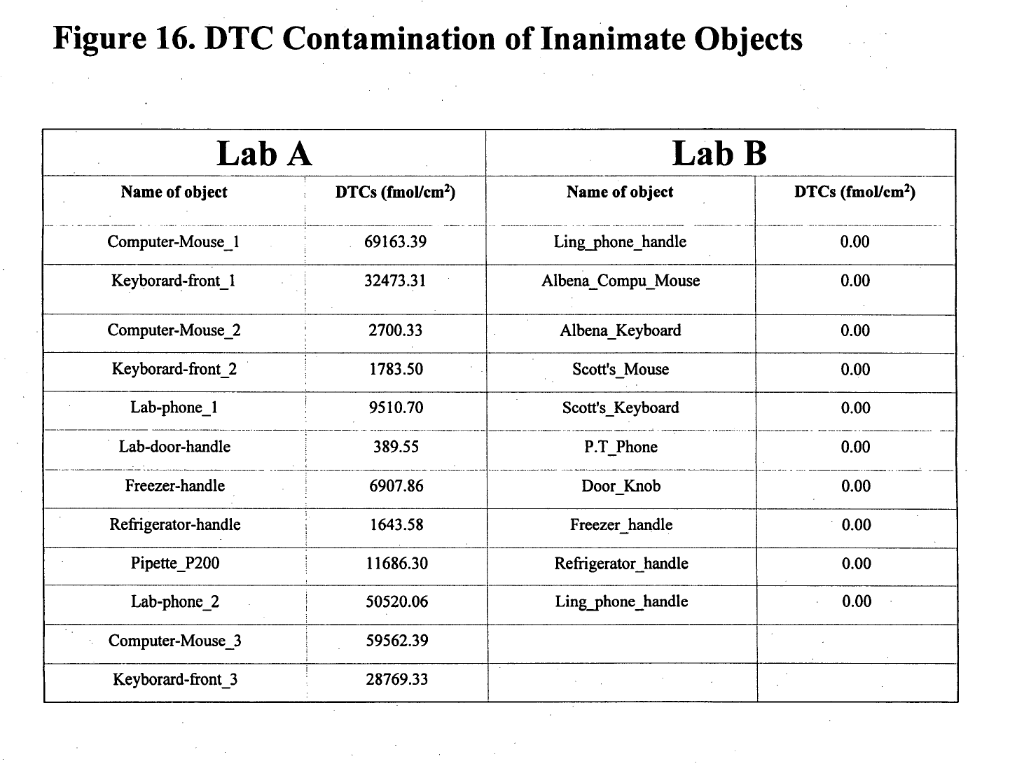

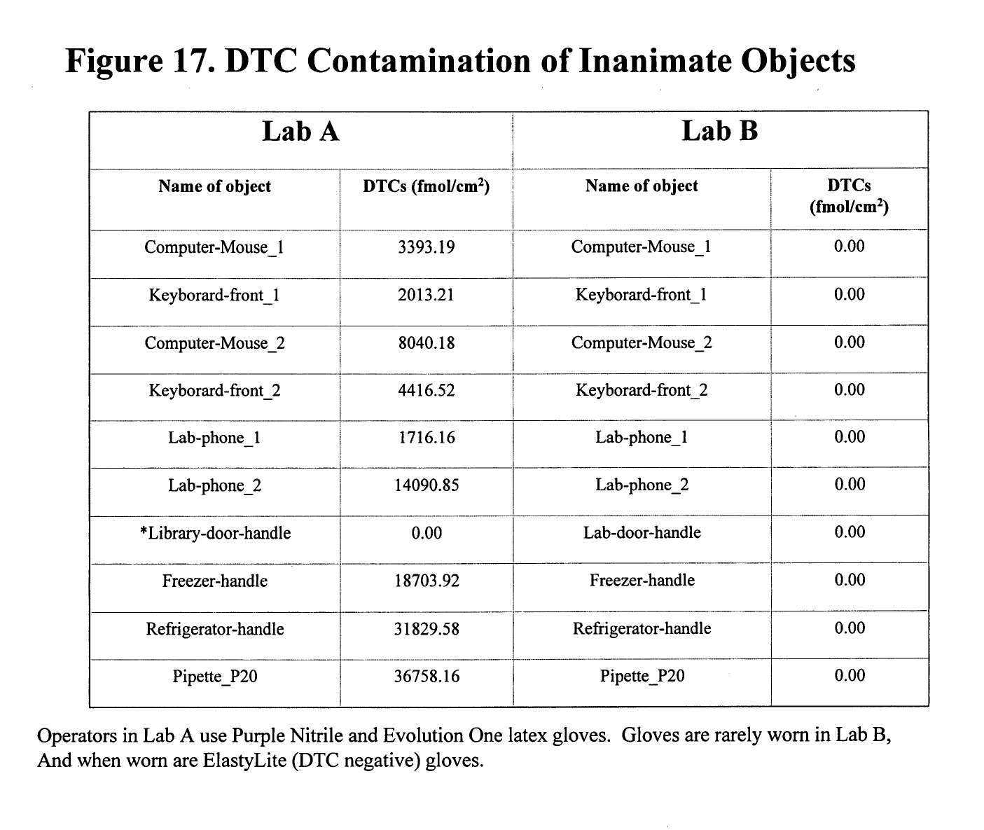

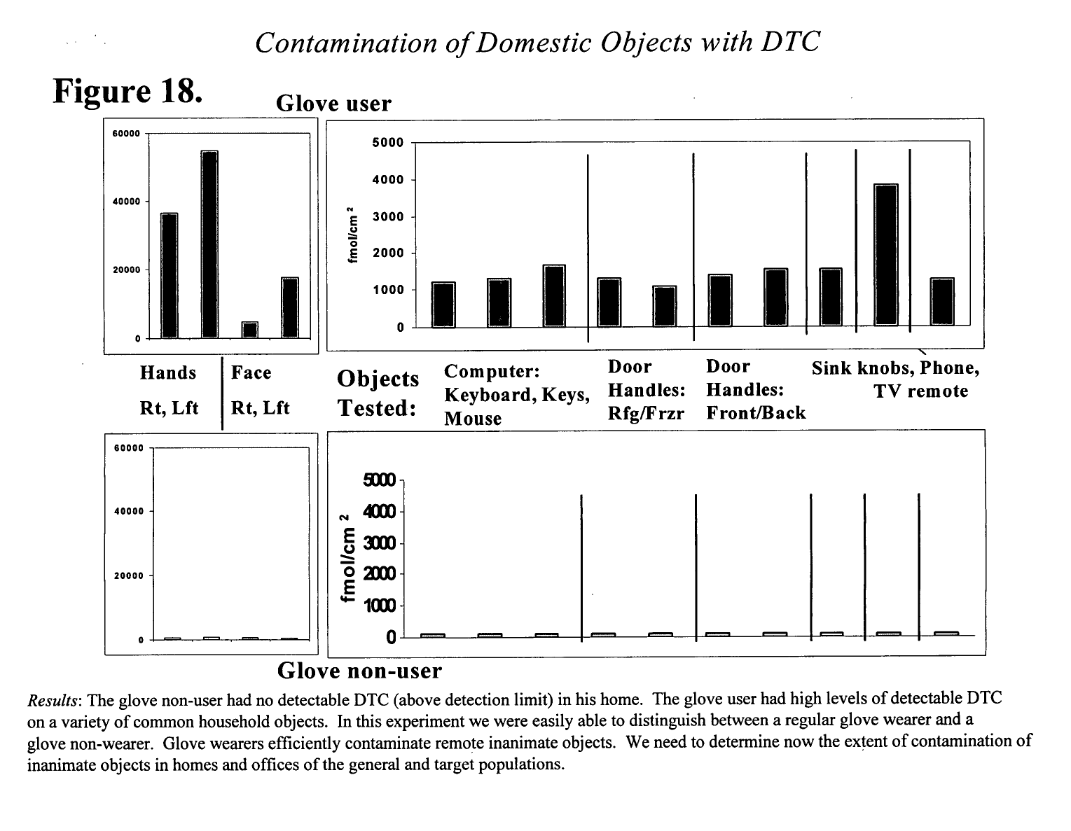

FIG. 12 illustrates that DTC can be transferred from a bare hand that has been exposed to rubber gloves to a person's face. FIG. 13 illustrates that DTC can be transferred from a washed, bare hand that has been exposed to rubber gloves to a person's face, although in smaller amounts than in FIG. 12. FIG. 14 illustrates that DTC can be transferred from a bare hand that has been exposed to rubber gloves to another person's hand by a handshake. Finally, FIG. 15 illustrates that DTC can be transferred from a bare hand that has been exposed to rubber gloves to an inanimate object. FIGS. 16-18 further illustrate how DTC is transferred from a glove wearer's hand to inanimate objects. FIG. 18 also illustrates how a non-glove wearer does not transfer DTC to inanimate objects.

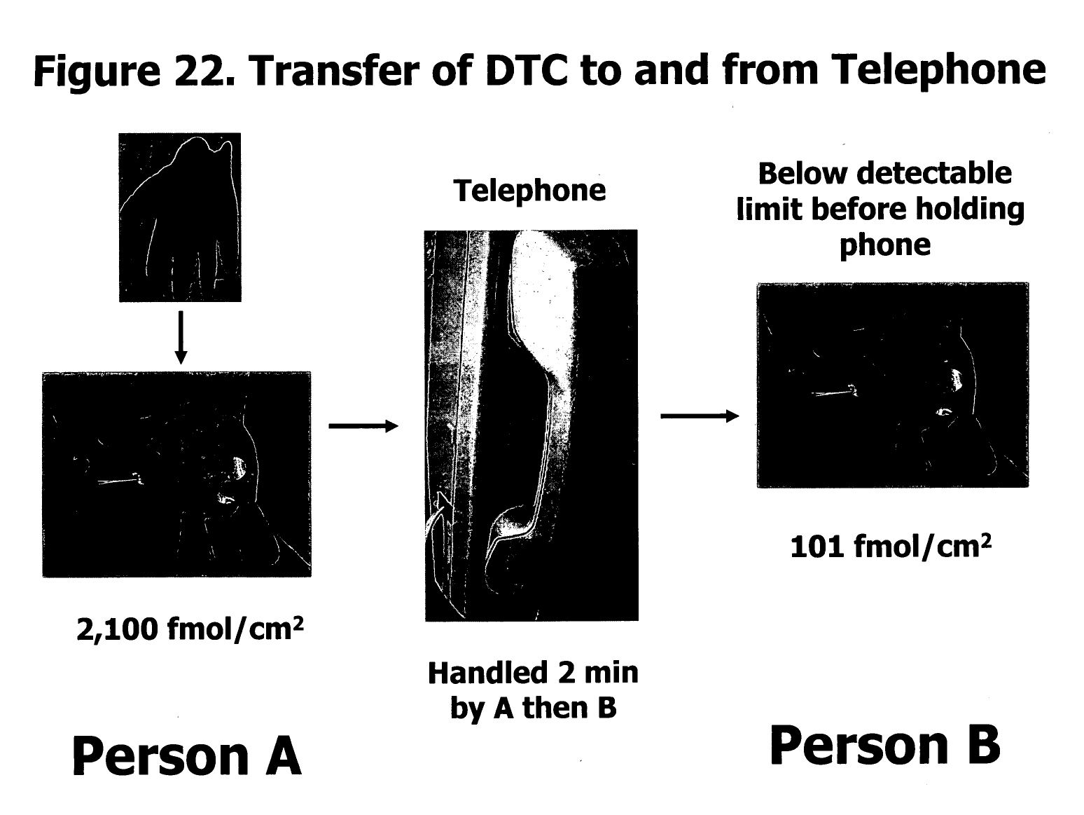

FIG. 22 illustrates how DTC can be transferred from a glove to a glove wearer's hand and how it can be subsequently transferred to an inanimate object, such as a telephone receiver. The subsequent transfer occurs when the glove wearer handles the receiver and has been observed when the contact lasts for about two minutes. Accordingly, a glove wearer's personal belongings can be tested to determine whether he or she recently came into contact with DTC containing materials.

DTC present on an inanimate object can also be transferred to a person who uses the object after a glove wearer. This permits identification of a chain of transfer, particularly if the DTC or other contaminant is unique or uniquely labeled. Generally, the level of DTC contamination on the second person's hand is less than the level on the hand of the original glove wearer. Therefore, the level of contamination can be used to determine whether a person came into direct contact or indirect contact with the DTC containing materials. A person who had direct contact with DTC containing materials, or other measurable contaminant, may be a terrorist or a criminal. A person who had indirect contact with DTC containing materials, or other measurable contaminant, may be a member of a criminal network that uses materials containing DTC or other contaminant.

A subject was tested for DTC contamination on one hand after wearing gloves containing DTC. The subject was tested by placing a D-Squame adhesive disc on different locations on the palm of the hand between five to ten times. The DTC level was found to be 2,100 fmol/cm². The subject then handled a telephone receiver for about two minutes and replaced it on the base. A second subject held the same telephone receiver in one hand and was tested for DTC contamination on that hand. Although the subject had below detectable levels of DTC on his hand prior to coming into contact with the telephone receiver, the subject tested positive for DTC contamination after touching the receiver. The DTC level was measured by placing a D-Squame adhesive disc different locations on the palm of the hand between five to ten times and was found to be 101 fmol/cm².

### Example 5

### Study of Multiple Discs vs. Multiple Applications of Single Disc

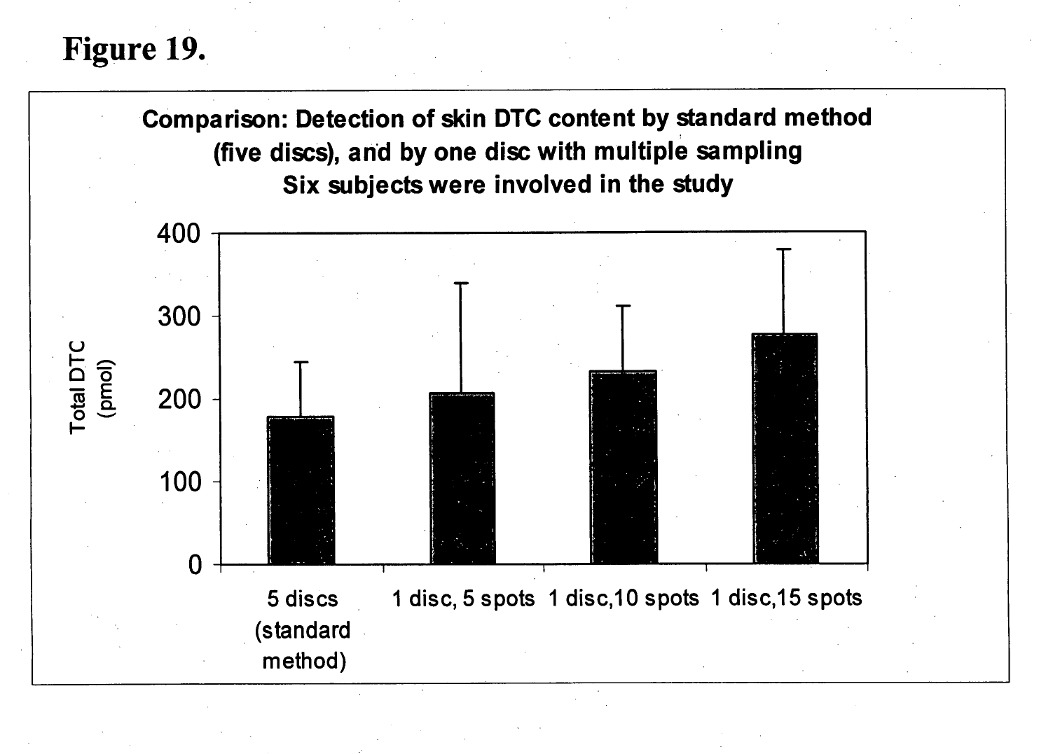

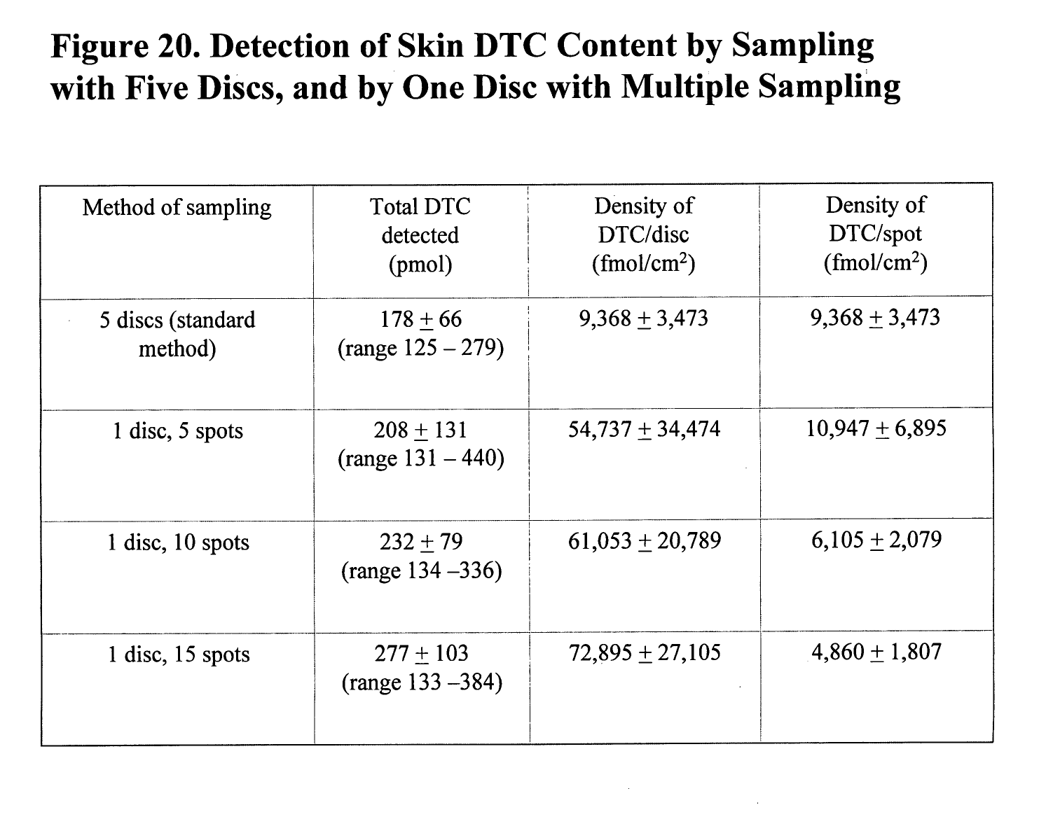

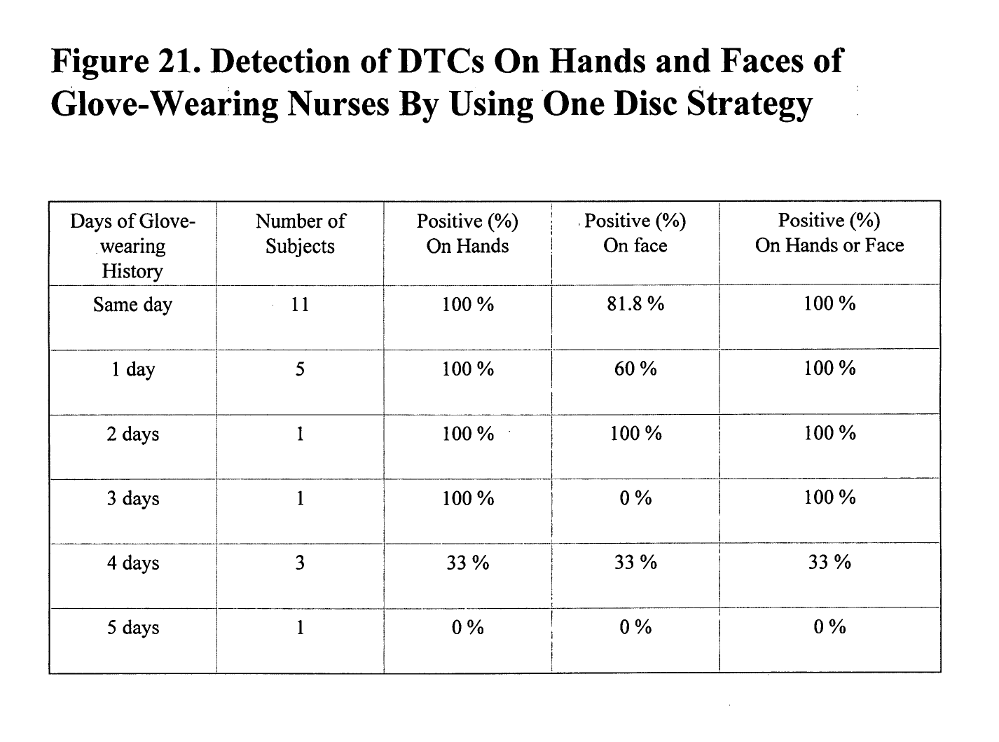

Five D-Squame adhesive discs (22 mm diameter; area 19 cm²) were applied to different locations on the skin surface of the hand to detect and measure DTC levels. Another subject had a single D-Squame adhesive disc (22 mm diameter; area 19 cm²) applied to five different locations on the skin surface of the hand to detect and measure DTC levels. The measurements were compared. FIGS. 19 and 20 illustrate that there is not much difference between using a single disc that is applied in five different locations and using five discs each applied in different locations. The total DTC began to level off after 5 samplings. FIG. 21 further illustrates the one disc method.

### Example 6

### Population Study

Extended population studies of DTC contamination on the faces and hands of subjects have confirmed that every individual who tested positive for contamination had a history of rubber glove use. Moreover, nearly every individual who tested positive on their hands also tested positive on their face. This is likely because individuals universally transfer contaminants, such as DTC, from their contaminated hands to their face. Moreover, these results suggest that facial contamination can be detected even where an individual has successfully decontaminated his or her hands.

Thirty two volunteer subjects were tested for DTC contamination. Six of the subjects tested positive for contamination while the remaining twenty-six subjects did not test positive for DTC. All six of the positive subjects reported recent glove use and all but one of the subjects had relatively low levels of contamination. The subject with high levels of contamination reported wearing gloves daily. Five of the six subjects also tested positive for facial contamination. Of the twenty-six subjects that tested negative, one reported using Playtex gloves once six to seven days earlier.

### Example 7

### PCR Test

The sample collecting device described herein also collects samples of a person's DNA from the skin. Accordingly, it provides a simple and convenient method of collecting DNA samples. It addition, it does not become easily contaminated.

A DNA sample was collected from the face of a subject using the device described herein by placing the adhesive disc on different locations of the cheek of the subject between five to ten times. The DNA was removed from the adhesive disc and extracted through the use of the Gentra Systems DNA extraction kit for mouse tail DNA. This kit is well-known by those skilled in the art and is a robust extraction kit. The extracted DNA was then reconstituted according to the instructions in the Gentra Systems DNA extraction kit, except that less hydration buffer was employed to make a more concentrated sample. Subsequently, the DNA sample was run on PCR using the human β-globin gene primers resulting in a 408 bp product. In addition, a human genomic control was used from Promega Corporation. The following PCR conditions were used as recommended by Takura Corporation of Japan:

### PCR Conditions

| | | |
|---|---|---|
| 94° C. | 1 minute | 1 cycle |
| 94° C. | 30 | 30 cycles |
| 55° C. | 1 minute | |
| 42° C. | 1 minute | |
| 4° C. | Hold | |

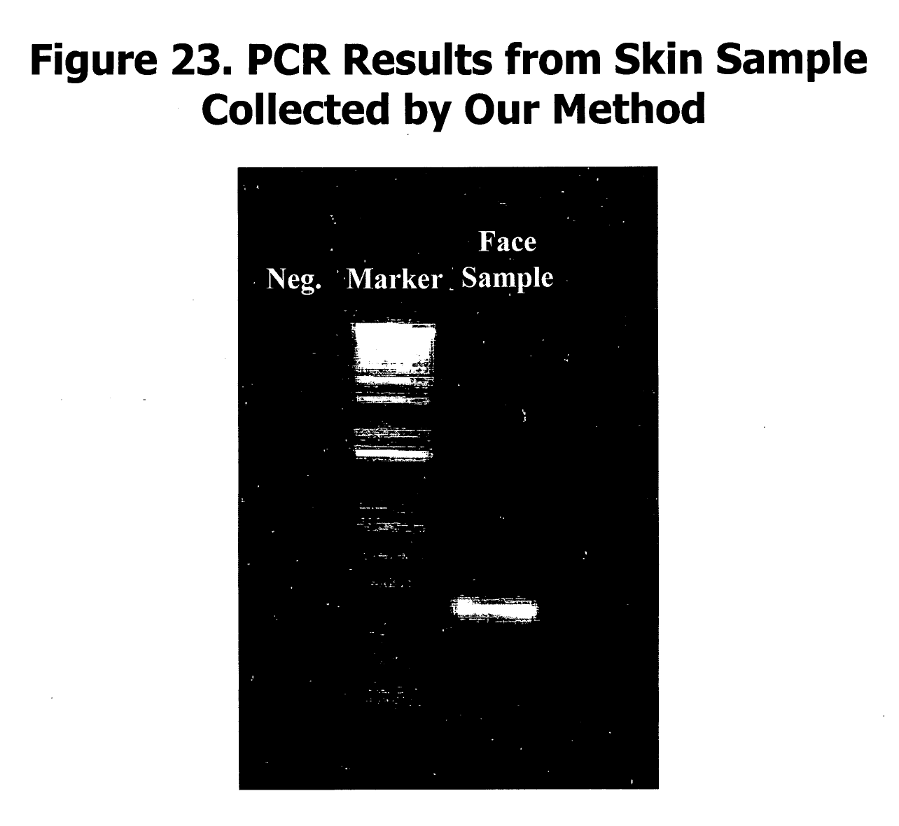

The PCR product was then applied to a gel and, as can be seen in FIG. 23, the 408 bp fragment was observed in a single clear band in the column labeled “Face Sample.” A negative control disc that did not contain any contaminant was also tested and applied to the gel. As can be seen in FIG. 23, the column labeled “Neg.” did not result in any bands. Finally, the marker control, or DNA ladder, resulted in a spectrum of bands across the gel.

### PREFERRED EMBODIMENTS

1. A device for collecting a sample from a test surface, wherein the sample comprises a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof, comprising:

a substrate;

a housing; and

optionally, a cap,

wherein the housing supports the substrate and wherein the cap protects the substrate from contamination.

2. The device according to claim 1, wherein the substrate is coated with an adhesive.

3. The device according to claim 1, wherein the sample is not collected from a plant, blood, urine, or internal tissue.

4. The device according to claim 1, wherein the test surface is selected from the group consisting of skin, clothing, luggage, or other inanimate object.

5. The device according to claim 4, wherein the skin is human skin.

6. The device according to claim 1, wherein the sample is used to detect the contaminant from the test surface.

7. The device according to claim 1, wherein the sample further comprises skin cells, skin oil, DNA, toxic agents, radiological agents, chemical agents, biological agents, or a combination thereof.

8. The device according to claim 7, wherein the sample is used to isolate and analyze DNA.

9. The device according to claim 7, wherein the sample is used to detect the toxic agent, the radiological agent, the chemical agent, the biological agent, or the combination thereof.

10. The device according to claim 7, wherein the toxic agent is selected from the group consisting of cytotoxins, diphtheria toxin, cholera toxin, ricin, 0-Shiga-like toxin, SLT-I, SLT-II, SLT-IIᵥ, LT toxin, C3 toxin, Shiga toxin, pertussis toxin, tetanus toxin, Pseudomonas exotoxin, alorin, saponin, modeccin, gelanin, and a combination thereof.

11. The device according to claim 7, wherein the radiological agent is selected from the group consisting of ²²⁴Ac, ²²⁵Ac, ¹¹¹Ag, ⁷²As, ⁷⁷As, ²¹¹At, ²¹⁷At, ¹⁹⁸Au, ¹⁹⁹Au, ²¹¹Bi, ²¹²Bi, ²¹³Bi, ⁷⁵Br, ⁷⁶Br, ⁷⁷Br, ^{80m}Br, ¹¹C, ⁵⁵Co, ⁵⁷Co, ⁵⁸Co, ⁵¹Cr, ⁶²Cu, ⁶⁴Cu, ⁶⁷Cu, ¹⁵²Dy, ¹⁶⁶Dy, ¹⁶⁹Er, ¹⁸F, ⁵²Fe, ⁵⁹Fe, ²⁵⁵Fm, ²²¹Fr, ⁶⁷Ga, ⁶⁸Ga, ¹⁵⁴⁻¹⁵⁸Gd, ¹⁰⁷Hg, ¹⁹⁷Hg, ²⁰³Hg, ¹⁶¹Ho, ¹⁶⁶Ho, ¹²⁰I, ¹²³I, ¹²⁴I, ¹²⁵I, ¹²⁶I, ¹³¹I, ¹³³I, ¹¹⁰In, ¹¹¹In, ^{113m}In, ^{114m}In, ¹⁹⁴Ir, ¹⁹²Ir, ¹⁷⁷Lu, ⁵¹Mn, ^{52m}Mn, ⁹⁹Mo, ¹³N, ¹⁵O, ^{189m}OS, ³²P, ³³P, ²¹¹Pb, ²¹²Pb, ¹⁰⁹Pd, ¹⁴⁹Pm, ²¹⁵Po, ¹⁴²Pr, ¹⁴³Pr, ¹⁰⁹Pt, ¹⁹⁷Pt, ²²³Ra, ²²⁶Ra, ^{82m}Rb, ¹⁸⁶Re, ¹⁸⁸Re, ¹⁸⁹Re, ¹⁰³Rh, ¹⁰⁵Rh, ²¹⁹Rn, ⁹⁵Ru, ⁹⁷Ru, ^{103m}Ru, ¹⁰⁵Ru, ¹¹⁹Sb, ⁷⁵Se, ¹⁵³Sm, ⁸³Sr, ⁸⁹Sr, ¹⁶¹Tb, ^{94m}Tc, ⁹⁴Tc, ^{99m}Tc, ^{121m}Te, ^{122m}Te, ^{125m}Te, ²⁰¹Tl, ¹⁶⁵Tm, ¹⁶⁷Tm, ¹⁶⁸Tm, ⁸⁶Y, ⁹⁰Y, ¹⁶⁹Yb, ⁸⁹Zr, and a combination thereof

12. The device according to claim 7, wherein the chemical agent is selected from the group consisting of Cyclohexyl Sarin, GE, Sarin, Soman, Tabun, VE, VG, V-Gas, VM, VX, Distilled Mustard, Lewisite, Nitrogen Mustard, Phosgene Oxime), Ethyldichloroarsine, Lewisite 1, Lewisite 2, Lewisite 3, Methyldichloroarsine, Mustard/Lewisite, Mustard/T, Nitrogen Mustard, Nitrogen Mustard, Phenodichloroarsine, and Sesqui Mustard, Arsine, Cyanogen Chloride, Hydrogen Chloride, Hydrogen Cyanide, Chlorine, Diphosgene, Cyanide, Nitrogen Oxide, Perflurorisobutylene, Phosgene, Red Phosphorous, Sulfur Trioxide-Chlorosulfonic Acid, Teflon and Perflurorisobutylene, Titanium Tetrachloride, Zinc Oxide, Agent 15, BZ, Cannabinoids, Fentanyls, LSD, Phenothiazines, Bromobenzylcyanide, Chloroacetophenone, Chloropicrin, CN in Benzene and Carbon Tetrachloride, CN in Chloroform, CN and Chloropicrin in Chloroform, CR, CS, Adamsite, Diphenylchloroarsine, Diphenylcyanoarsine, opiods, opiates, narcotics, drugs of abuse, and a combination thereof.

13. The device according to claim 7, wherein the biological agent is selected from the group consisting of Anthrax, Cholera, Plague, Botulism, Tularemia, Variola, Q Fever, Staphylococcal Enterotoxin B, Brucellosis, Venezuelan Equine Encephalitis, Tricothecene, Viral hemorrhagic fevers, Filoviruses, Ebola, Marburg, Arenaviruses, Lassa, Machupo, Nipah virus, Melioidosis, Psittacosis, Typhus fever, Viral encephalitis, Alphaviruses, Venezuelan equine encephalitis, Eastern equine encephalitis, Western equine encephalitis, Water safety threats, Hantaviruses, Multidrug-resistant tuberculosis, Nipah virus, Tickborne encephalitis viruses, Tickborne hemorrhagic fever viruses, Yellow fever, and a combination thereof.

14. The device according to claim 1, wherein the contaminant is from protective gear.

15. The device according to claim 14, wherein the protective gear comprises a rubber glove, a rubber apron, a rubber mask, or a rubber boot.

16. The device according to claim 2, wherein the adhesive comprises a polymer.

17. The device according to claim 1, wherein the substrate comprises a polymer.

18. The device according to claim 17, wherein the polymer comprises polyester.

19. The device according to claim 1, wherein the substrate comprises a disc.

20. The device according to claim 19, wherein the disc is about 22-mm in diameter.

21. The device according to claim 1, wherein the housing comprises a polymer.

22. The device according to claim 21, wherein the polymer comprises delron, polyethylene, or polypropylene.

23. The device according to claim 1, wherein the housing comprises a screw mechanism or a stamp mechanism that allows the substrate to protrude beyond the surface of the housing and to be withdrawn into the housing.

24. The device according to claim 1, wherein the cap comprises a snap-on cap or a screw-on cap.

25. The device according to claim 2, further comprising a film that covers the adhesive.

26. A method for collecting a sample from a test surface, wherein the sample comprises a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof comprising:

providing a device comprising a substrate; a housing; and, optionally, a cap, wherein the housing supports the substrate and wherein the cap protects the substrate from contamination;
optionally, advancing the substrate to protrude from the housing;
exposing the substrate;
contacting the substrate with the test surface one or more times;
optionally, withdrawing the substrate into the housing; and
enclosing the substrate.

27. The method according to claim 26, wherein the substrate is coated with an adhesive.

28. The method according to claim 26, wherein the test surface is selected from the group consisting of skin, clothing, luggage, or other inanimate object.

29. The method according to claim 28, wherein the skin is human skin.

30. The method according to claim 26, wherein the sample is used to detect the contaminant from the test surface.

31. The method according to claim 26, wherein the sample further comprises skin cells, skin oil, DNA, toxic agents, radiological agents, chemical agents, biological agents, or a combination thereof.

32. The method according to claim 31, wherein the sample is used to isolate and analyze DNA.

33. The method according to claim 31, wherein the sample is used to detect the toxic agent, the radiological agent, the chemical agent, the biological agent, or the combination thereof.

34. The method according to claim 31, wherein the toxic agent is selected from the group consisting of cytotoxins, diphtheria toxin, cholera toxin, ricin, 0-Shiga-like toxin, SLT-I, SLT-II, SLT-IIᵥ, LT toxin, C3 toxin, Shiga toxin, pertussis toxin, tetanus toxin, Pseudomonas exotoxin, alorin, saponin, modeccin, gelanin, and a combination thereof.

35. The method according to claim 31, wherein the radiological agent is selected from the group consisting of ²²⁴Ac, ²²⁵Ac, ¹¹¹Ag, ⁷²As, ⁷⁷A, ²¹¹At, ²¹⁷At, ¹⁹⁸Au, ¹⁹⁹Au, ²¹¹Bi, ²¹²Bi, ²¹³Bi, ⁷⁵Br, ⁷⁶Br, ⁷⁷Br, ^{80m}Br, ¹¹C, ⁵⁵Co, ⁵⁷Co, ⁵⁸Co, ⁵¹Cr, ⁶²Cu, ⁶⁴Cu, ⁶⁷Cu, ¹⁵²Dy, ¹⁶⁶Dy, ¹⁶⁹Er, ¹⁸F, ⁵²Fe, ⁵⁹Fe, ²⁵⁵Fm, ²²¹Fr, ⁶⁷Ga, ⁶⁸Ga, ¹⁵⁴⁻¹⁵⁸Gd, ¹⁰⁷Hg, ¹⁹⁷Hg, ²⁰³Hg, ¹⁶¹Ho, ¹⁶⁶Ho, ¹²⁰I, ¹²³I, ¹²⁴I, ¹²⁵I, ¹²⁶I, ¹³¹I, ¹³³I, ¹¹⁰In, ¹¹¹In, ^{113m}In, ^{114m}In, ¹⁹⁴Ir, ¹⁹²Ir, ¹⁷⁷Lu, ⁵¹Mn, ^{52m}Mn, ⁹⁹Mo, ¹³N, ¹⁵O, ^{189m}Os, ³²P, ³³P, ²¹¹Pb, ²¹²Pb, ¹⁰⁹Pd, ¹⁴⁹Pm, ²¹⁵Po, ¹⁴²Pr, ¹⁴³Pr, ¹⁰⁹Pt, ¹⁹⁷Pt, ²²³Ra, ²²⁶Ra, ^{82m}Rb, ¹⁸⁶Re, ¹⁸⁸Re, ¹⁸⁹Re, ¹⁰³Rh, ¹⁰⁵Rh, ²¹⁹Rn, ⁹⁵Ru, ⁹⁷Ru, ^{103m}Ru, ¹⁰⁵Ru, ¹¹⁹Sb, ⁷⁵Se, ¹⁵³Sm, ⁸³Sr, ⁸⁹Sr, ¹⁶¹Tb, ^{94m}Tc, ⁹⁴Tc, ^{99m}Tc, ^{121m}Te, ^{122m}Te, ^{125m}Te, ²⁰¹Tl, ¹⁶⁵Tm, ¹⁶⁷Tm, ¹⁶⁸Tm, ⁸⁶Y, ⁹⁰Y, ¹⁶⁹Yb, ⁸⁹Zr, and a combination thereof.

36. The method according to claim 31, wherein the chemical agent is selected from the group consisting of Cyclohexyl Sarin, GE, Sarin, Soman, Tabun, VE, VG, V-Gas, VM, VX, Distilled Mustard, Lewisite, Nitrogen Mustard, Phosgene Oxime), Ethyldichloroarsine, Lewisite 1, Lewisite 2, Lewisite 3, Methyldichloroarsine, Mustard/Lewisite, Mustard/T, Nitrogen Mustard, Nitrogen Mustard, Phenodichloroarsine, and Sesqui Mustard, Arsine, Cyanogen Chloride, Hydrogen Chloride, Hydrogen Cyanide, Chlorine, Diphosgene, Cyanide, Nitrogen Oxide, Perflurorisobutylene, Phosgene, Red Phosphorous, Sulfur Trioxide-Chlorosulfonic Acid, Teflon and Perflurorisobutylene, Titanium Tetrachloride, Zinc Oxide, Agent 15, BZ, Cannabinoids, Fentanyls, LSD, Phenothiazines, Bromobenzylcyanide, Chloroacetophenone, Chloropicrin, CN in Benzene and Carbon Tetrachloride, CN in Chloroform, CN and Chloropicrin in Chloroform, CR, CS, Adamsite, Diphenylchloroarsine, Diphenylcyanoarsine, opiods, opiates, narcotics, drugs of abuse, and a combination thereof.

37. The method according to claim 31, wherein the biological agent is selected from the group consisting of Anthrax, Cholera, Plague, Botulism, Tularemia, Variola, Q Fever, Staphylococcal Enterotoxin B, Brucellosis, Venezuelan Equine Encephalitis, Tricothecene, Viral hemorrhagic fevers, Filoviruses, Ebola, Marburg, Arenaviruses, Lassa, Machupo, Nipah virus, Melioidosis, Psittacosis, Typhus fever, Viral encephalitis, Alphaviruses, Venezuelan equine encephalitis, Eastern equine encephalitis, Western equine encephalitis, Water safety threats, Hantaviruses, Multidrug-resistant tuberculosis, Nipah virus, Tickbome encephalitis viruses, Tickbome hemorrhagic fever viruses, Yellow fever, and a combination thereof.

38. The method according to claim 26, wherein the contaminant is from protective gear.

39. The method according to claim 38, wherein the protective gear comprises a rubber glove, a rubber apron, a rubber mask, or a rubber boot.

40. The method according to claim 27, wherein the adhesive comprises a polymer.

41. The method according to claim 26, wherein the substrate comprises a polymer.

42. The method according to claim 41, wherein the polymer comprises polyester.

43. The method according to claim 26, wherein the substrate comprises a disc.

44. The method according to claim 43, wherein the disc is about 22-mm in diameter.

45. The method according to claim 26, wherein the housing comprises a polymer.

46. The method according to claim 45, wherein the polymer comprises delron, polyethylene, or polypropylene.

47. The method according to claim 26, wherein the housing comprises a screw mechanism or a stamp mechanism that allows the substrate to protrude beyond the surface of the housing and to be withdrawn into the housing.

48. The method according to claim 26, wherein the cap comprises a snap-on cap or a screw-on cap.

49. The method according to claim 26, wherein the substrate is exposed by removing the cap and is enclosed by replacing the cap.

50. The method according to claim 27, wherein the device further comprises a film that covers the adhesive.

51. The method according to claim 50, wherein the substrate is exposed by removing the film and is enclosed by placing the device in a sterile and sealable bag, container, or pouch.

52. A method for detecting a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof comprising:

contacting a sample collected from a test surface comprising the contaminant with a cyclocondensation reactant;

allowing the contaminant to react with the cyclocondensation reactant; and

detecting a cyclocondensation product with HPLC.

53. The method according to claim 52, wherein the sample is collected according to claim 26.

54. The method according to claim 52, further comprising contacting an adhesive with the test surface one or more times to collect the sample and, optionally, removing the sample from the adhesive.

55. The method according to claim 54, wherein the adhesive coats a substrate that is supported by a housing and, optionally, the adhesive is enclosed with a cap.

56. The method according to claim 54, wherein the test surface is selected from the group consisting of skin, clothing, luggage, or other inanimate object.

57. The method according to claim 56, wherein the skin is human skin.

58. The method according to claim 54, wherein the adhesive comprises a polymer.

59. The method according to claim 55, wherein the substrate comprises a polymer.

60. The method according to claim 59, wherein the polymer comprises polyester.

61. The method according to claim 55, wherein the substrate comprises a disc.

62. The method according to claim 61, wherein the disc is about 22-mm in diameter.

63. The method according to claim 55, wherein the housing comprises a polymer.

64. The method according to claim 63, wherein the polymer comprises delron, polyethylene, or polypropylene.

65. The method according to claim 55, wherein the housing comprises a screw mechanism or a stamp mechanism that allows the substrate to protrude beyond the surface of the housing and to be withdrawn into the housing.

66. The method according to claim 55, wherein the cap comprises a snap-on cap or a screw-on cap.

67. The method according to claim 54, wherein the adhesive is covered by a film.

68. The method according to claim 52, wherein the sample further comprises skin cells, skin oil, DNA, toxic agents, radiological agents, chemical agents, biological agents, or a combination thereof.

69. The method according to claim 68, further comprising isolating DNA and analyzing the DNA.

70. The method according to claim 68, further comprising detecting the toxic agent, the radiological agent, the chemical agent, the biological agent, or the combination thereof.

71. The method according to claim 68, wherein the toxic agent is selected from the group consisting of cytotoxins, diphtheria toxin, cholera toxin, ricin, 0-Shiga-like toxin, SLT-I, SLT-II, SLT-IIᵥ, LT toxin, C3 toxin, Shiga toxin, pertussis toxin, tetanus toxin, Pseudomonas exotoxin, alorin, saponin, modeccin, gelanin, and a combination thereof.

72. The method according to claim 68, wherein the radiological agent is selected from the group consisting of ²²⁴Ac, ²²⁵Ac, ¹¹¹Ag, ⁷²As, ⁷⁷As, ²¹¹At, ²¹⁷At, ¹⁹⁸Au, ¹⁹⁹Au, ²¹¹Bi, ²¹²Bi, ²¹³Bi, ⁷⁵Br, ⁷⁶Br, ⁷⁷Br, ^{80m}Br, ¹¹C, ⁵⁵Co, ⁵⁷Co, ⁵⁸Co, ⁵¹Cr, ⁶²Cu, ⁶⁴Cu, ⁶⁷Cu, ¹⁵²Dy, ¹⁶⁶Dy, ¹⁶⁹Er, ¹⁸F, ⁵²Fe, ⁵⁹Fe, ²⁵⁵Fm, ²²¹Fr, ⁶⁷Ga, ⁶⁸Ga, ¹⁵⁴⁻¹⁵⁸Gd, ¹⁰⁷Hg, ¹⁹⁷Hg, ²⁰³Hg, ¹⁶¹Ho, ¹⁶⁶Ho, ¹²⁰I, ¹²³I, ¹²⁴I, ¹²⁵I, ¹²⁶I, ¹³¹I, ¹³³I, ¹¹⁰In, ¹¹¹In, ^{113m}In, ^{114m}In, ¹⁹⁴Ir, ¹⁹²Ir, ¹⁷⁷Lu, ⁵¹Mn, ^{52m}Mn, ⁹⁹Mo, ¹³N, ¹⁵O, ^{189m}Os, ³²P, ³³P, ²¹¹Pb, ²¹²Pb, ¹⁰⁹Pd, ¹⁴⁹PM, ²¹⁵Po, ¹⁴²Pr, ¹⁴³Pr, ¹⁰⁹Pt, ¹⁹⁷Pt, ²²³Ra, ²²⁶Ra, ^{82m}Rb, ¹⁸⁶Re, ¹⁸⁸Re, ¹⁸⁹Re, ¹⁰³Rh, ¹⁰⁵Rh, ²¹⁹Rn, ⁹⁵Ru, ⁹⁷Ru, ^{103m}Ru, ¹⁰⁵Ru, ¹¹⁹Sb, ⁷⁵Se, ¹⁵³Sm, ⁸³Sr, ⁸⁹Sr, ¹⁶¹Tb, ^{94m}Tc, ⁹⁴Tc, ^{99m}Tc, ^{121m}Te, ^{122m}Te, ^{125m}Te, ²⁰¹Tl, ¹⁶⁵Tm, ¹⁶⁷Tm, ¹⁶⁸Tm, ⁸⁶Y, ⁹⁰Y, ¹⁶⁹Yb, ⁸⁹Zr, and a combination thereof.

73. The method according to claim 68, wherein the chemical agent is selected from the group consisting of Cyclohexyl Sarin, GE, Sarin, Soman, Tabun, VE, VG, V-Gas, VM, VX, Distilled Mustard, Lewisite, Nitrogen Mustard, Phosgene Oxime), Ethyldichloroarsine, Lewisite 1, Lewisite 2, Lewisite 3, Methyldichloroarsine, Mustard/Lewisite, Mustard/T, Nitrogen Mustard, Nitrogen Mustard, Phenodichloroarsine, and Sesqui Mustard, Arsine, Cyanogen Chloride, Hydrogen Chloride, Hydrogen Cyanide, Chlorine, Diphosgene, Cyanide, Nitrogen Oxide, Perflurorisobutylene, Phosgene, Red Phosphorous, Sulfur Trioxide-Chlorosulfonic Acid, Teflon and Perflurorisobutylene, Titanium Tetrachloride, Zinc Oxide, Agent 15, BZ, Cannabinoids, Fentanyls, LSD, Phenothiazines, Bromobenzylcyanide, Chloroacetophenone, Chloropicrin, CN in Benzene and Carbon Tetrachloride, CN in Chloroform, CN and Chloropicrin in Chloroform, CR, CS, Adamsite, Diphenylchloroarsine, Diphenylcyanoarsine, opiods, opiates, narcotics, drugs of abuse, and a combination thereof.

74. The method according to claim 68, wherein the biological agent is selected from the group consisting of Anthrax, Cholera, Plague, Botulism, Tularemia, Variola, Q Fever, Staphylococcal Enterotoxin B, Brucellosis, Venezuelan Equine Encephalitis, Tricothecene, Viral hemorrhagic fevers, Filoviruses, Ebola, Marburg, Arenaviruses, Lassa, Machupo, Nipah virus, Melioidosis, Psittacosis, Typhus fever, Viral encephalitis, Alphaviruses, Venezuelan equine encephalitis, Eastern equine encephalitis, Western equine encephalitis, Water safety threats, Hantaviruses, Multidrug-resistant tuberculosis, Nipah virus, Tickborne encephalitis viruses, Tickborne hemorrhagic fever viruses, Yellow fever, and a combination thereof.

75. The method according to claim 52, wherein the contaminant is from protective gear.

76. The method according to claim 75, wherein the protective gear comprises a rubber glove, a rubber apron, a rubber mask, or a rubber boot.

77. The method of claim 52, wherein the cyclocondensation reactant is selected from the group consisting of 1,2-benzenedithiol, 3,4-dimercaptotoluene, and 2,3-dimercaptopropanol.

78. The method of claim 52, wherein the cyclocondensation product is 1,3-benzodithiole-2-thione.

79. The method of claim 52, wherein the sample is collected in the field.

80. The method of claim 52, wherein the method is used to determine the efficiency of decontamination of a surface.

81. An assay for detecting a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof comprising:

providing a device comprising a substrate; a housing; and, optionally, a cap, wherein the housing supports the substrate and wherein the cap protects the substrate from contamination;

contacting the substrate with a test surface one or more times to collect a sample comprising the contaminant;

optionally, removing the contaminant from the substrate; and

detecting the contaminant.

82. The assay according to claim 81, wherein the substrate is coated with an adhesive.

83. The assay according to claim 81, wherein the test surface is selected from the group consisting of skin, clothing, luggage, or other inanimate object.

84. The assay according to claim 83, wherein the skin is human skin.

85. The assay according to claim 81, wherein the sample further comprises skin cells, skin oil, DNA, toxic agents, radiological agents, chemical agents, biological agents, or a combination thereof.

86. The assay according to claim 85, further comprising isolating DNA and analyzing the DNA.

87. The assay according to claim 85, further comprising detecting the toxic agent, the radiological agent, the chemical agent, the biological agent, or the combination thereof.

88. The assay according to claim 85, wherein the toxic agent is selected from the group consisting of cytotoxins, diphtheria toxin, cholera toxin, ricin, 0-Shiga-like toxin, SLT-I, SLT-II, SLT-IIᵥ, LT toxin, C3 toxin, Shiga toxin, pertussis toxin, tetanus toxin, Pseudomonas exotoxin, alorin, saponin, modeccin, gelanin, and a combination thereof.

89. The assay according to claim 85, wherein the radiological agent is selected from the group consisting of ²²⁴Ac, ²²⁵Ac, ¹¹¹Ag, ⁷²As, ⁷⁷As, ²¹¹At, ²¹⁷At, ¹⁹⁸Au, ¹⁹⁹Au, ²¹¹Bi, ²¹²Bi, ²¹³Bi, ⁷⁵Br, ⁷⁶Br, ⁷⁷Br, ^{80m}Br, ¹¹C, ⁵⁵Co, ⁵⁷Co, ⁵⁸Co, ⁵¹Cr, ⁶²Cu, ⁶⁴Cu, ⁶⁷Cu, ¹⁵²Dy, ¹⁶⁶Dy, ¹⁶⁹Er, ¹⁸F, ⁵²Fe, ⁵⁹Fe, ²⁵⁵Fm, ²²¹Fr, ⁶⁷Ga, ⁶⁸Ga, ¹⁵⁴⁻¹⁵⁸Gd, ¹⁰⁷Hg, ¹⁹⁷Hg, ²⁰³Hg, ¹⁶¹Ho, ¹⁶⁶Ho, ¹²⁰I, ¹²³I, ¹²⁴I, ¹²⁵I, ¹²⁶I, ¹³¹I, ¹³³I, ¹¹⁰In, ¹¹¹In, ^{113m}In, ^{114m}In, ¹⁹⁴Ir, ¹⁹²Ir, ¹⁷⁷Lu, ⁵¹Mn, ^{52m}Mn, ⁹⁹Mo, ¹³N, ¹⁵O, ^{189m}Os, ³²P, ³³P, ²¹¹Pb, ²¹²Pb, ¹⁰⁹Pd, ¹⁴⁹Pm, ²¹⁵Po, ¹⁴²Pr, ¹⁴³Pr, ¹⁰⁹Pt, ¹⁹⁷Pt, ²²³Ra, ²²⁶Ra, ^{82m}Rb, ¹⁸⁶Re, ¹⁸⁸Re, ¹⁸⁹Re, ¹⁰³Rh, ¹⁰⁵Rh, ²¹⁹Rn, ⁹⁵Ru, ⁹⁷Ru, ^{103m}Ru, ¹⁰⁵Ru, ¹¹⁹Sb, ⁷⁵Se, ¹⁵³Sm, ⁸³Sr, ⁸⁹Sr, ¹⁶¹Tb, ^{94m}Tc, ⁹⁴Tc, ^{99m}Tc, ^{121m}Te, ^{122m}Te, ^{125m}Te, ²⁰¹Tl, ¹⁶⁵Tm, ¹⁶⁷Tm, ¹⁶⁸Tm, ⁸⁶Y, ⁹⁰Y, ¹⁶⁹Yb, ⁸⁹Zr, and a combination thereof

90. The assay according to claim 85, wherein the chemical agent is selected from the group consisting of Cyclohexyl Sarin, GE, Sarin, Soman, Tabun, VE, VG, V-Gas, VM, VX, Distilled Mustard, Lewisite, Nitrogen Mustard, Phosgene Oxime), Ethyldichloroarsine, Lewisite 1, Lewisite 2, Lewisite 3, Methyldichloroarsine, Mustard/Lewisite, Mustard/T, Nitrogen Mustard, Nitrogen Mustard, Phenodichloroarsine, and Sesqui Mustard, Arsine, Cyanogen Chloride, Hydrogen Chloride, Hydrogen Cyanide, Chlorine, Diphosgene, Cyanide, Nitrogen Oxide, Perflurorisobutylene, Phosgene, Red Phosphorous, Sulfur Trioxide-Chlorosulfonic Acid, Teflon and Perflurorisobutylene, Titanium Tetrachloride, Zinc Oxide, Agent 15, BZ, Cannabinoids, Fentanyls, LSD, Phenothiazines, Bromobenzylcyanide, Chloroacetophenone, Chloropicrin, CN in Benzene and Carbon Tetrachloride, CN in Chloroform, CN and Chloropicrin in Chloroform, CR, CS, Adamsite, Diphenylchloroarsine, Diphenylcyanoarsine, opiods, opiates, narcotics, drugs of abuse, and a combination thereof.

91. The assay according to claim 85, wherein the biological agent is selected from the group consisting of Anthrax, Cholera, Plague, Botulism, Tularemia, Variola, Q Fever, Staphylococcal Enterotoxin B, Brucellosis, Venezuelan Equine Encephalitis, Tricothecene, Viral hemorrhagic fevers, Filoviruses, Ebola, Marburg, Arenaviruses, Lassa, Machupo, Nipah virus, Melioidosis, Psittacosis, Typhus fever, Viral encephalitis, Alphaviruses, Venezuelan equine encephalitis, Eastern equine encephalitis, Western equine encephalitis, Water safety threats, Hantaviruses, Multidrug-resistant tuberculosis, Nipah virus, Tickborne encephalitis viruses, Tickborne hemorrhagic fever viruses, Yellow fever, and a combination thereof.

92. The assay according to claim 81, wherein the contaminant is from protective gear.

93. The assay according to claim 92, wherein the protective gear comprises a rubber glove, a rubber apron, a rubber mask, or a rubber boot.

94. The assay according to claim 82, wherein the adhesive comprises a polymer.

95. The assay according to claim 81, wherein the substrate comprises a polymer.

96. The assay according to claim 95, wherein the polymer comprises polyester.

97. The assay according to claim 81, wherein the substrate comprises a disc.

98. The assay according to claim 97, wherein the disc is about 22-mm in diameter.

99. The assay according to claim 81, wherein the housing comprises a polymer.

100. The assay according to claim 99, wherein the polymer comprises delron, polyethylene, or polypropylene.

101. The assay according to claim 81, wherein the housing comprises a screw mechanism or a stamp mechanism that allows the substrate to protrude beyond the surface of the housing and to be withdrawn into the housing.

102. The assay according to claim 81, wherein the cap comprises a snap-on cap or a screw-on cap.

103. The assay according to claim 82, wherein the device further comprises a film that covers the adhesive.

104. The assay of claim 81, wherein the detection step comprises:
reacting the contaminant with a cyclocondensation reactant; and
detecting a cyclocondensation product with HPLC.

105. The assay of claim 104, wherein the cyclocondensation reactant is selected from the group consisting of 1,2-benzenedithiol, 3,4-dimercaptotoluene, and 2,3-dimercaptopropanol.

106. The assay of claim 104, wherein the cyclocondensation product is 1,3-benzodithiole-2-thione.

107. The assay of claim 81, wherein the sample is collected in the field.

108. The assay of claim 81, wherein the method is assay to determine the efficiency of decontamination of a surface.

109. A method for identifying a suspect who has come into contact with protective gear comprising:

collecting a sample from a person's skin with a device comprising a substrate; a housing; and, optionally, a cap, wherein the housing supports the substrate and wherein the cap protects the substrate from contamination, by contacting the substrate with the skin one or more times;

optionally, removing the sample from the substrate; and

detecting a contaminant in the sample.

110. The method according to claim 109, wherein the substrate is coated with an adhesive.

111. The method according to claim 109, wherein the suspect is a criminal or a terrorist.

112. The method according to claim 109, wherein the contaminant is selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof.

113. The method according to claim 112, wherein the sample further comprises skin cells, skin oil, DNA, toxic agents, radiological agents, chemical agents, biological agents, or a combination thereof.

114. The method according to claim 113, further comprising isolating DNA and analyzing the DNA.

115. The method according to claim 113, further comprising detecting the toxic agent, the radiological agent, the chemical agent, the biological agent, or the combination thereof.

116. The method according to claim 113, wherein the toxic agent is selected from the group consisting of cytotoxins, diphtheria toxin, cholera toxin, ricin, 0-Shiga-like toxin, SLT-I, SLT-II, SLT-IIᵥ, LT toxin, C3 toxin, Shiga toxin, pertussis toxin, tetanus toxin, Pseudomonas exotoxin, alorin, saponin, modeccin, gelanin, and a combination thereof.

117. The method according to claim 113, wherein the radiological agent is selected from the group consisting of ²²⁴Ac, ²²⁵Ac, ¹¹¹Ag, ⁷²As, ⁷⁷As, ²¹¹At, ²¹⁷At, ¹⁹⁸Au, ¹⁹⁹Au, ²¹¹Bi, ²¹²Bi, ²¹³Bi, ⁷⁵Br, ⁷⁶Br, ⁷⁷Br, ^{80m}Br, ¹¹C, ⁵⁵Co, ⁵⁷Co, ⁵⁸Co, ⁵¹Cr, ⁶²Cu, ⁶⁴Cu, ⁶⁷Cu, ¹⁵²Dy, ¹⁶⁶Dy, ¹⁶⁹Er, ¹⁸F, ⁵²Fe, ⁵⁹Fe, ²⁵⁵Fm, ²²¹Fr, ⁶⁷Ga, ⁶⁸Ga, ¹⁵⁴⁻¹⁵⁸Gd, ¹⁰⁷Hg, ¹⁹⁷Hg, ²⁰³Hg, ¹⁶¹Ho, ¹⁶⁶Ho, ¹²⁰I, ¹²³I, ¹²⁴I, ¹²⁵I, ¹²⁶I, ¹³¹I, ¹³³I, ¹¹⁰In, ¹¹¹In, ^{113m}In, ^{114m}In, ¹⁹⁴Ir, ¹⁹²Ir, ¹⁷⁷Lu, ⁵¹Mn, ^{52m}Mn, ⁹⁹Mo, ¹³N, ¹⁵O, ^{189m}Os, ³²P, ³³P, ²¹¹Pb, ²¹²Pb, ¹⁰⁹Pd, ¹⁴⁹ Pm, ²¹⁵Po, ¹⁴²Pr, ¹⁴³Pr, ¹⁰⁹Pt, ¹⁹⁷Pt, ²²³Ra, ²²⁶Ra, ^{82m}Rb, ¹⁸⁶Re, ¹⁸⁸Re, ¹⁸⁹Re, ¹⁰³Rh, ¹⁰⁵Rh, ²¹⁹Rn, ⁹⁵Ru, ⁹⁷Ru, ^{103m}Ru, ¹⁰⁵Ru, ¹¹⁹Sb, ⁷⁵Se, ¹⁵³Sm, ⁸³Sr, ⁸⁹Sr, ¹⁶¹Tb, ^{94m}Tc, ⁹⁴Tc, ^{99m}Tc, ^{121m}Te, ^{122m}Te, ^{125m}Te, ²⁰¹Tl, ¹⁶⁵Tm, ¹⁶⁷Tm, ¹⁶⁸Tm, ⁸⁶Y, ⁹⁰Y, ¹⁶⁹Yb, ⁸⁹Zr, and a combination thereof.

118. The method according to claim 113, wherein the chemical agent is selected from the group consisting of Cyclohexyl Sarin, GE, Sarin, Soman, Tabun, VE, VG, V-Gas, VM, VX, Distilled Mustard, Lewisite, Nitrogen Mustard, Phosgene Oxime), Ethyldichloroarsine, Lewisite 1, Lewisite 2, Lewisite 3, Methyldichloroarsine, Mustard/Lewisite, Mustard/T, Nitrogen Mustard, Nitrogen Mustard, Phenodichloroarsine, and Sesqui Mustard, Arsine, Cyanogen Chloride, Hydrogen Chloride, Hydrogen Cyanide, Chlorine, Diphosgene, Cyanide, Nitrogen Oxide, Perflurorisobutylene, Phosgene, Red Phosphorous, Sulfur Trioxide-Chlorosulfonic Acid, Teflon and Perflurorisobutylene, Titanium Tetrachloride, Zinc Oxide, Agent 15, BZ, Cannabinoids, Fentanyls, LSD, Phenothiazines, Bromobenzylcyanide, Chloroacetophenone, Chloropicrin, CN in Benzene and Carbon Tetrachloride, CN in Chloroform, CN and Chloropicrin in Chloroform, CR, CS, Adamsite, Diphenylchloroarsine, Diphenylcyanoarsine, opiods, opiates, narcotics, drugs of abuse, and a combination thereof.

119. The method according to claim 113, wherein the biological agent is selected from the group consisting of Anthrax, Cholera, Plague, Botulism, Tularemia, Variola, Q Fever, Staphylococcal Enterotoxin B, Brucellosis, Venezuelan Equine Encephalitis, Tricothecene, Viral hemorrhagic fevers, Filoviruses, Ebola, Marburg, Arenaviruses, Lassa, Machupo, Nipah virus, Melioidosis, Psittacosis, Typhus fever, Viral encephalitis, Alphaviruses, Venezuelan equine encephalitis, Eastern equine encephalitis, Western equine encephalitis, Water safety threats, Hantaviruses, Multidrug-resistant tuberculosis, Nipah virus, Tickborne encephalitis viruses, Tickborne hemorrhagic fever viruses, Yellow fever, and a combination thereof.

120. The method according to claim 109, wherein the protective gear comprises a rubber glove, a rubber apron, a rubber mask, or a rubber boot.

121. The method according to claim 110, wherein the adhesive comprises a polymer.

122. The method according to claim 109, wherein the substrate comprises a polymer.

123. The method according to claim 122, wherein the polymer comprises polyester.

124. The method according to claim 109, wherein the substrate comprises a disc.

125. The method according to claim 124, wherein the disc is about 22-mm in diameter.

126. The method according to claim 109, wherein the housing comprises a polymer.

127. The method according to claim 126, wherein the polymer comprises delron, polyethylene, or polypropylene.

128. The method according to claim 109, wherein the housing comprises a screw mechanism or a stamp mechanism that allows the substrate to protrude beyond the surface of the housing and to be withdrawn into the housing.

129. The method according to claim 109, wherein the cap comprises a snap-on cap or a screw-on cap.

130. The method according to claim 110, wherein the device further comprises a film that covers the adhesive.

131. The method according to claim 109, wherein the detection step comprises:

contacting the sample or the substrate with a cyclocondensation reactant; and

detecting the presence or absence of a cyclocondensation product with HPLC.

132. The method of claim 131, wherein the cyclocondensation reactant is selected from the group consisting of 1,2-benzenedithiol, 3,4-dimercaptotoluene, and 2,3-dimercaptopropanol.

133. The method of claim 131, wherein the cyclocondensation product is 1,3-benzodithiole-2-thione.

134. The method of claim 109, wherein the sample is collected in the field.

135. A method of forensic analysis for determining if a suspect has contacted protective gear comprising:

collecting a sample from the suspect's skin with a device comprising a substrate; a housing; and, optionally, a cap, wherein the housing supports the substrate and wherein the cap protects the substrate from contamination, by contacting the substrate with the skin one or more times;

optionally, removing the sample from the substrate; and

detecting the presence or absence of a contaminant in the sample.

136. The method according to claim 135, wherein the substrate is coated with an adhesive.

137. The method according to claim 135, wherein the suspect is a criminal or a terrorist.

138. The method according to claim 135, wherein the contaminant is selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof.

139. The method according to claim 138, wherein the sample further comprises skin cells, skin oil, DNA, toxic agents, radiological agents, chemical agents, biological agents, or a combination thereof.

140. The method according to claim 139, further comprising isolating DNA and analyzing the DNA.

141. The method according to claim 139, further comprising detecting the toxic agent, the radiological agent, the chemical agent, the biological agent, or the combination thereof.

142. The method according to claim 139, wherein the toxic agent is selected from the group consisting of cytotoxins, diphtheria toxin, cholera toxin, ricin, 0-Shiga-like toxin, SLT-I, SLT-II, SLT-IIᵥ, LT toxin, C3 toxin, Shiga toxin, pertussis toxin, tetanus toxin, Pseudomonas exotoxin, alorin, saponin, modeccin, gelanin, and a combination thereof.

143. The method according to claim 139, wherein the radiological agent is selected from the group consisting of ²²⁴Ac, ²²⁵Ac, ¹¹¹Ag, ⁷²As, ⁷⁷As, ²¹¹At, ²¹⁷At, ¹⁹⁸ Au, ¹⁹⁹Au, ²¹¹Bi, ²¹²Bi, ²¹³Bi, ⁷⁵Br, ⁷⁶Br, ⁷⁷Br, ^{80m}Br, ¹¹C, ⁵⁵Co, ⁵⁷Co, ⁵⁸Co, ⁵¹Cr, ⁶²Cu, ⁶⁴Cu, ⁶⁷Cu, ¹⁵²Dy, ¹⁶⁶Dy, ¹⁶⁹Er, ¹⁸F, ⁵²Fe, ⁵⁹Fe, ²⁵⁵Fm, ²²¹Fr, ⁶⁷Ga, ⁶⁸Ga, ¹⁵⁴⁻¹⁵⁸Gd, ¹⁰⁷Hg, ¹⁹⁷Hg, ²⁰³Hg, ¹⁶¹Ho, ¹⁶⁶Ho, ¹²⁰I, ¹²³I, ¹²⁴I, ¹²⁵I, ¹²⁶I, ¹³¹I, ¹³³I, ¹¹⁰In, ¹¹¹In, ^{113m}In, ^{114m}In, ¹⁹⁴Ir, ¹⁹²Ir, ¹⁷⁷Lu, ⁵¹Mn, ^{52m}Mn, ⁹⁹Mo, ¹³N, ¹⁵O, ^{189m}Os, ³²P, ³³P, ²¹¹Pb, ²¹²Pb, ¹⁰⁹Pd, ¹⁴⁹ Pm, ²¹⁵Po, ¹⁴²Pr, ¹⁴³Pr, ¹⁰⁹Pt, ¹⁹⁷Pt, ²²³Ra, ²²⁶Ra, ^{82m}Rb, ¹⁸⁶Re, ¹⁸⁸Re, ¹⁸⁹Re, ¹⁰³Rh, ¹⁰⁵Rh, ²¹⁹Rn, ⁹⁵Ru, ⁹⁷Ru, ^{103m}Ru, ¹⁰⁵Ru, ¹¹⁹Sb, ⁷⁵Se, ¹⁵³Sm, ⁸³Sr, ⁸⁹Sr, ¹⁶¹Tb, ^{94m}Tc, ⁹⁴Tc, ^{99m}Tc, ^{121m}Te, ^{122m}Te, ^{125m}Te, ²⁰¹Tl, ¹⁶⁵Tm, ¹⁶⁷Tm, ¹⁶⁸Tm, ⁸⁶Y, ⁹⁰Y, ¹⁶⁹Yb, ⁸⁹Zr, and a combination thereof.

144. The method according to claim 139, wherein the chemical agent is selected from the group consisting of Cyclohexyl Sarin, GE, Sarin, Soman, Tabun, VE, VG, V-Gas, VM, VX, Distilled Mustard, Lewisite, Nitrogen Mustard, Phosgene Oxime), Ethyldichloroarsine, Lewisite 1, Lewisite 2, Lewisite 3, Methyldichloroarsine, Mustard/Lewisite, Mustard/T, Nitrogen Mustard, Nitrogen Mustard Phenodichloroarsine, and Sesqui Mustard, Arsine, Cyanogen Chloride, Hydrogen Chloride, Hydrogen Cyanide, Chlorine, Diphosgene, Cyanide, Nitrogen Oxide, Perflurorisobutylene, Phosgene, Red Phosphorous, Sulfur Trioxide-Chlorosulfonic Acid, Teflon and Perflurorisobutylene, Titanium Tetrachloride, Zinc Oxide, Agent 15, BZ, Cannabinoids, Fentanyls, LSD, Phenothiazines, Bromobenzylcyanide, Chloroacetophenone, Chloropicrin, CN in Benzene and Carbon Tetrachloride, CN in Chloroform, CN and Chloropicrin in Chloroform, CR, CS, Adamsite, Diphenylchloroarsine, Diphenylcyanoarsine, opiods, opiates, narcotics, drugs of abuse, and a combination thereof.

145. The method according to claim 139, wherein the biological agent is selected from the group consisting of Anthrax, Cholera, Plague, Botulism, Tularemia, Variola, Q Fever, Staphylococcal Enterotoxin B, Brucellosis, Venezuelan Equine Encephalitis, Tricothecene, Viral hemorrhagic fevers, Filoviruses, Ebola, Marburg, Arenaviruses, Lassa, Machupo, Nipah virus, Melioidosis, Psittacosis, Typhus fever, Viral encephalitis, Alphaviruses, Venezuelan equine encephalitis, Eastern equine encephalitis, Western equine encephalitis, Water safety threats, Hantaviruses, Multidrug-resistant tuberculosis, Nipah virus, Tickborne encephalitis viruses, Tickborne hemorrhagic fever viruses, Yellow fever, and a combination thereof.

146. The method according to claim 139, wherein the protective gear comprises a rubber glove, a rubber apron, a rubber mask, or a rubber boot.

147. The method according to claim 136, wherein the adhesive comprises a polymer.

148. The method according to claim 135, wherein the substrate comprises a polymer.

149. The method according to claim 148, wherein the polymer comprises polyester.

150. The method according to claim 135, wherein the substrate comprises a disc.

151. The method according to claim 150, wherein the disc is about 22-mm in diameter.

152. The method according to claim 135, wherein the housing comprises a polymer.

153. The method according to claim 152, wherein the polymer comprises delron, polyethylene, or polypropylene.

154. The method according to claim 135, wherein the housing comprises a screw mechanism or a stamp mechanism that allows the substrate to protrude beyond the surface of the housing and to be withdrawn into the housing.

155. The method according to claim 135, wherein the cap comprises a snap-on cap or a screw-on cap.

156. The method according to claim 136, wherein the device further comprises a film that covers the adhesive.

157. The method of claim 133, wherein the detection step comprises:

contacting the sample or the substrate with a cyclocondensation reactant; and

detecting the presence or absence of a cyclocondensation product with HPLC.

158. The method of claim 157, wherein the cyclocondensation reactant is selected from the group consisting of 1,2-benzenedithiol, 3,4-dimercaptotoluene, and 2,3-dimercaptopropanol.

159. The method of claim 157, wherein the cyclocondensation product is 1,3-benzodithiole-2-thione.

160. The method of claim 135, wherein the sample is collected in the field.

161. A method of identifying members of a criminal network comprising:

introducing protective gear with a contaminant into the network;

collecting a sample comprising the contaminant from a suspected member's skin with a device comprising a substrate; a housing; and, optionally, a cap, wherein the housing supports the substrate and wherein the cap protects the substrate from contamination, by contacting the substrate with the skin one or more times; and

detecting the presence or absence of the contaminant.

162. The method according to claim 161, wherein the substrate is coated with an adhesive.

163. The method according to claim 161, wherein the contaminant is selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof.

164. The method according to claim 163, wherein the sample further comprises skin cells, skin oil, DNA, toxic agents, radiological agents, chemical agents, biological agents, or a combination thereof.

165. The method according to claim 164, further comprising isolating DNA and analyzing the DNA.

166. The method according to claim 164, further comprising detecting the toxic agent, the radiological agent, the chemical agent, the biological agent, or the combination thereof.

167. The method according to claim 164, wherein the toxic agent is selected from the group consisting of cytotoxins, diphtheria toxin, cholera toxin, ricin, 0-Shiga-like toxin, SLT-I, SLT-II, SLT-IIᵥ, LT toxin, C3 toxin, Shiga toxin, pertussis toxin, tetanus toxin, Pseudomonas exotoxin, alorin, saponin, modeccin, gelanin, and a combination thereof.

168. The method according to claim 164, wherein the radiological agent is selected from the group consisting of ²²⁴Ac, ²²⁵Ac, ¹¹¹Ag, ⁷²As, ⁷⁷As, ²¹¹At, ²¹⁷At, ¹⁹⁸ Au, ¹⁹⁹Au, ²¹¹Bi, ²¹²Bi, ²¹³Bi, ⁷⁵Br, ⁷⁶Br, ⁷⁷Br, ^{80m}Br, ¹¹C, ⁵⁵CO, ⁵⁷Co, ⁵⁸Co, ⁵¹Cr, ⁶²Cu, ⁶⁴Cu, ⁶⁷Cu, ¹⁵²Dy, ¹⁶⁶Dy, ¹⁶⁹Er, ¹⁸F, ⁵²Fe, ⁵⁹Fe, ²⁵⁵Fm, ²²¹Fr, ⁶⁷Ga, ⁶⁸Ga, ¹⁵⁴⁻¹⁵⁸Gd, ¹⁰⁷Hg, ¹⁹⁷Hg, ²⁰³Hg, ¹⁶¹Ho, ¹⁶⁶Ho, ¹²⁰I, ¹²³I, ¹²⁴I, ¹²⁵I, ¹²⁶I, ¹³¹I, ¹³³I, ¹¹⁰In, ¹¹¹In, ^{113m}In, ^{114m}In, ¹⁹⁴Ir, ¹⁹²Ir, ¹⁷⁷Lu, ⁵¹Mn, ^{52m}Mn, ⁹⁹Mo, ¹³N, ¹⁵O, ^{89m}Os, ³²P, ³³P, ²¹¹Pb, ²¹²Pb, ¹⁰⁹Pd, ¹⁴⁹ Pm, ²¹⁵Po, ¹⁴²Pr, ¹⁴³Pr, ¹⁰⁹Pt, ¹⁹⁷Pt, ²²³Ra, ²²⁶Ra, ^{82m}Rb, ¹⁸⁶Re, ¹⁸⁸Re, ¹⁸⁹Re, ¹⁰³Rh, ¹⁰⁵Rh, ²¹⁹Rn, ⁹⁵Ru, ⁹⁷Ru, ^{103m}Ru, ¹⁰⁵Ru, ¹¹⁹Sb, ⁷⁵Se, ¹⁵³Sm, ⁸³Sr, ⁸⁹Sr, ¹⁶¹Tb, ^{94m}Tc, ⁹⁴Tc, ^{99m}Tc, ^{121m}Te, ^{122m}Te, ^{125m}Te, ²⁰¹Tl, ¹⁶⁵Tm, ¹⁶⁷Tm, ¹⁶⁸Tm, ⁸⁶Y, ⁹⁰Y, ¹⁶⁹Yb, ⁸⁹Zr, and a combination thereof.

169. The method according to claim 164, wherein the chemical agent is selected from the group consisting of Cyclohexyl Sarin, GE, Sarin, Soman, Tabun, VE, VG, V-Gas, VM, VX, Distilled Mustard, Lewisite, Nitrogen Mustard, Phosgene Oxime), Ethyldichloroarsine, Lewisite 1, Lewisite 2, Lewisite 3, Methyldichloroarsine, Mustard/Lewisite, Mustard/T, Nitrogen Mustard, Nitrogen Mustard, Phenodichloroarsine, and Sesqui Mustard, Arsine, Cyanogen Chloride, Hydrogen Chloride, Hydrogen Cyanide, Chlorine, Diphosgene, Cyanide, Nitrogen Oxide, Perflurorisobutylene, Phosgene, Red Phosphorous, Sulfur Trioxide-Chlorosulfonic Acid, Teflon and Perflurorisobutylene, Titanium Tetrachloride, Zinc Oxide, Agent 15, BZ, Cannabinoids, Fentanyls, LSD, Phenothiazines, Bromobenzylcyanide, Chloroacetophenone, Chloropicrin, CN in Benzene and Carbon Tetrachloride, CN in Chloroform, CN and Chloropicrin in Chloroform, CR, CS, Adamsite, Diphenylchloroarsine, Diphenylcyanoarsine, opiods, opiates, narcotics, drugs of abuse, and a combination thereof.

170. The method according to claim 164, wherein the biological agent is selected from the group consisting of Anthrax, Cholera, Plague, Botulism, Tularemia, Variola, Q Fever, Staphylococcal Enterotoxin B, Brucellosis, Venezuelan Equine Encephalitis, Tricothecene, Viral hemorrhagic fevers, Filoviruses, Ebola, Marburg, Arenaviruses, Lassa, Machupo, Nipah virus, Melioidosis, Psittacosis, Typhus fever, Viral encephalitis, Alphaviruses, Venezuelan equine encephalitis, Eastern equine encephalitis, Western equine encephalitis, Water safety threats, Hantaviruses, Multidrug-resistant tuberculosis, Nipah virus, Tickborne encephalitis viruses, Tickborne hemorrhagic fever viruses, Yellow fever, and a combination thereof.

171. The method according to claim 161, wherein the protective gear comprises a rubber glove, a rubber apron, a rubber mask, or a rubber boot.

172. The method according to claim 162, wherein the adhesive comprises a polymer.

173. The method according to claim 161, wherein the substrate comprises a polymer.

174. The method according to claim 173, wherein the polymer comprises polyester.

175. The method according to claim 161, wherein the substrate comprises a disc.

176. The method according to claim 175, wherein the disc is about 22-mm in diameter.

177. The method according to claim 161, wherein the housing comprises a polymer.

178. The method according to claim 177, wherein the polymer comprises delron, polyethylene, or polypropylene.

179. The method according to claim 161, wherein the housing comprises a screw mechanism or a stamp mechanism that allows the substrate to protrude beyond the surface of the housing and to be withdrawn into the housing.

180. The method according to claim 161, wherein the cap comprises a snap-on cap or a screw-on cap.

181. The method according to claim 162, wherein the device further comprises a film that covers the adhesive.

182. The method according to claim 161, wherein the detection step comprises:

contacting the sample with a cyclocondensation reactant;

allowing the contaminant to react with the cyclocondensation reactant; and

detecting the presence or absence of a cyclocondensation product with HPLC.

183. The method of claim 182, wherein the cyclocondensation reactant is selected from the group consisting of 1,2-benzenedithiol, 3,4-dimercaptotoluene, and 2,3-dimercaptopropanol.

184. The method of claim 182, wherein the cyclocondensation product is 1,3-benzodithiole-2-thione.

185. The method according to claim 161, wherein the detection step comprises detecting the presence or absence of the contaminant with a spectrophotometer.

186. The method of claim 161, wherein the sample is collected in the field.

187. A kit comprising:

a device for collecting a sample from a test surface, comprising a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof comprising a substrate; a housing; and, optionally, a cap, wherein the housing supports the substrate and wherein the cap protects the substrate from contamination.

188. The kit according to claim 187, wherein the substrate is coated with an adhesive.

189. The kit according to claim 187, wherein the sample is not collected from a plant, blood, urine, or internal tissue.

190. The kit according to claim 187, wherein the test surface is selected from the group consisting of skin, fruit, vegetables, clothing, luggage, or other inanimate object.

191. The kit according to claim 190, wherein the skin is human skin.

192. The kit according to claim 180, wherein the sample is used to detect the contaminant from the test surface.

193. The kit according to claim 180, wherein the sample further comprises skin cells, skin oil, DNA, toxic agents, radiological agents, chemical agents, biological agents, or a combination thereof.

194. The kit according to claim 193, wherein the sample is used to isolate and analyze DNA.

195. The kit according to claim 193, wherein the sample is used to detect the toxic agent, the radiological agent, the chemical agent, the biological agent, or the combination thereof.

196. The kit according to claim 193, wherein the toxic agent is selected from the group consisting of cytotoxins, diphtheria toxin, cholera toxin, ricin, 0-Shiga-like toxin, SLT-I, SLT-II, SLT-IIᵥ, LT toxin, C3 toxin, Shiga toxin, pertussis toxin, tetanus toxin, Pseudomonas exotoxin, alorin, saponin, modeccin, gelanin, and a combination thereof.

197. The kit according to claim 193, wherein the radiological agent is selected from the group consisting of ²²⁴Ac, ²²⁵Ac, ¹¹¹Ag, ⁷²As, ⁷⁷As, ²¹¹At, ²¹⁷At, ¹⁹⁸Au, ¹⁹⁹Au, ²¹¹Bi, ²¹²Bi, ²¹³Bi, ⁷⁵Br, ⁷⁶Br, ⁷⁷Br, ^{80m}Br, ¹¹C, ⁵⁵Co, ⁵⁷Co, ⁵⁸Co, ⁵¹Cr, ⁶²Cu, ⁶⁴Cu, ⁶⁷Cu, ¹⁵²Dy, ¹⁶⁶Dy, ¹⁶⁹Er, ¹⁸F, ⁵²Fe, ⁵⁹Fe, ²⁵⁵Fm, ²²¹Fr, ⁶⁷Ga, ⁶⁸Ga, ¹⁵⁴⁻¹⁵⁸Gd, ¹⁰⁷Hg, ¹⁹⁷Hg, ²⁰³Hg, ¹⁶¹Ho, ¹⁶⁶Ho, ¹²⁰I, ¹²³I, ¹²⁴I, ¹²⁵I, ¹²⁶I, ¹³¹I, ¹³³I, ¹¹⁰In, ¹¹¹In, ^{113m}In, ^{114m}In, ¹⁹⁴Ir, ¹⁹²Ir, ¹⁷⁷Lu, ⁵¹Mn, ^{52m}Mn, ⁹⁹Mo, ¹³N, ¹⁵O, ^{189m}Os, ³²P, ³³P, ²¹¹Pb, ²¹²Pb, ¹⁰⁹Pd, ¹⁴⁹Pm, ²¹⁵Po, ¹⁴²Pr, ¹⁴³Pr, ¹⁰⁹Pt, ¹⁹⁷Pt, ²²³Ra, ²²⁶Ra, ^{82m}Rb, ¹⁸⁶Re, ¹⁸⁸Re, ¹⁸⁹Re, ¹⁰³Rh, ¹⁰⁵Rh, ²¹⁹Rn, ⁹⁵Ru, ⁹⁷Ru, ^{103m}Ru, ¹⁰⁵Ru, ¹¹⁹Sb, ⁷⁵Se, ¹⁵³Sm, ⁸³Sr, ⁸⁹Sr, ¹⁶¹Tb, ^{94m}Tc, ⁹⁴Tc, ^{99m}Tc, ^{121m}Te, ^{122m}Te, ^{125m}Te, ²⁰¹Tl, ¹⁶⁵Tm, ¹⁶⁷Tm, ¹⁶⁸Tm, ⁸⁶Y, ⁹⁰Y, ¹⁶⁹Yb, ⁸⁹Zr, and a combination thereof.

198. The kit according to claim 193, wherein the chemical agent is selected from the group consisting of Cyclohexyl Sarin, GE, Sarin, Soman, Tabun, VE, VG, V-Gas, VM, VX, Distilled Mustard, Lewisite, Nitrogen Mustard, Phosgene Oxime), Ethyldichloroarsine, Lewisite 1, Lewisite 2, Lewisite 3, Methyldichloroarsine, Mustard/Lewisite, Mustard/T, Nitrogen Mustard, Nitrogen Mustard, Phenodichloroarsine, and Sesqui Mustard, Arsine, Cyanogen Chloride, Hydrogen Chloride, Hydrogen Cyanide, Chlorine, Diphosgene, Cyanide, Nitrogen Oxide, Perflurorisobutylene, Phosgene, Red Phosphorous, Sulfur Trioxide-Chlorosulfonic Acid, Teflon and Perflurorisobutylene, Titanium Tetrachloride, Zinc Oxide, Agent 15, BZ, Cannabinoids, Fentanyls, LSD, Phenothiazines, Bromobenzylcyanide, Chloroacetophenone, Chloropicrin, CN in Benzene and Carbon Tetrachloride, CN in Chloroform, CN and Chloropicrin in Chloroform, CR, CS, Adamsite, Diphenylchloroarsine, Diphenylcyanoarsine, opiods, opiates, narcotics, drugs of abuse, and a combination thereof.

199. The kit according to claim 193, wherein the biological agent is selected from the group consisting of Anthrax, Cholera, Plague, Botulism, Tularemia, Variola, Q Fever, Staphylococcal Enterotoxin B, Brucellosis, Venezuelan Equine Encephalitis, Tricothecene, Viral hemorrhagic fevers, Filoviruses, Ebola, Marburg, Arenaviruses, Lassa, Machupo, Nipah virus, Melioidosis, Psittacosis, Typhus fever, Viral encephalitis, Alphaviruses, Venezuelan equine encephalitis, Eastern equine encephalitis, Western equine encephalitis, Water safety threats, Hantaviruses, Multidrug-resistant tuberculosis, Nipah virus, Tickborne encephalitis viruses, Tickborne hemorrhagic fever viruses, Yellow fever, and a combination thereof.

200. The kit according to claim 187, wherein the contaminant is from protective gear.

201. The kit according to claim 200, wherein the protective gear comprises a rubber glove, a rubber apron, a rubber mask, or a rubber boot.

202. The kit according to claim 188, wherein the adhesive comprises a polymer.

203. The kit according to claim 187, wherein the substrate comprises a polymer.

204. The kit according to claim 203, wherein the polymer comprises polyester.

205. The kit according to claim 187, wherein the substrate comprises a disc.

206. The kit according to claim 205, wherein the disc is about 22-mm in diameter.

207. The kit according to claim 187, wherein the housing comprises a polymer.

208. The kit according to claim 207, wherein the polymer comprises delron, polyethylene, or polypropylene.

209. The kit according to claim 187, wherein the housing comprises a screw mechanism or a stamp mechanism that allows the substrate to protrude beyond the surface of the housing and to be withdrawn into the housing.

210. The kit according to claim 187, wherein the cap comprises a snap-on cap or a screw-on cap.

211. The kit according to claim 188, further comprising a film that covers the adhesive.

212. The kit according to claim 187, further comprising a portable HPLC.

What is claimed is:

1. A device for collecting a sample from a test surface, wherein the sample comprises a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof, comprising:
a substrate;
a housing; and
optionally, a cap,
wherein the housing supports the substrate and wherein the cap protects the substrate from contamination.

2. The device according to claim 1, wherein the substrate is coated with an adhesive.

3. The device according to claim 1, wherein the sample is not collected from a plant, blood, urine, or internal tissue.

4. The device according to claim 1, wherein the test surface is selected from the group consisting of skin, clothing, luggage, or other inanimate object.

5. The device according to claim 4, wherein the skin is human skin.

6. The device according to claim 1, wherein the sample is used to detect the contaminant from the test surface.

7. The device according to claim 1, wherein the sample further comprises skin cells, skin oil, DNA, toxic agents, radiological agents, chemical agents, biological agents, or a combination thereof.

8. The device according to claim 7, wherein the sample is used to isolate and analyze DNA.

9. The device according to claim 7, wherein the sample is used to detect the toxic agent, the radiological agent, the chemical agent, the biological agent, or the combination thereof.

10. The device according to claim 7, wherein the toxic agent is selected from the group consisting of cytotoxins, diphtheria toxin, cholera toxin, ricin, 0-Shiga-like toxin, SLT-I, SLT-II, SLT-IIᵥ, LT toxin, C3 toxin, Shiga toxin, pertussis toxin, tetanus toxin, Pseudomonas exotoxin, alorin, saponin, modeccin, gelanin, and a combination thereof.

11. The device according to claim 7, wherein the radiological agent is selected from the group consisting of ²²⁴Ac, ²²⁵Ac, ¹¹¹Ag, ⁷²As, ⁷⁷As, ²¹¹At, ²¹⁷At, ¹⁹⁸Au, ¹⁹⁹Au, ²¹¹Bi, ²¹²Bi, ²¹³Bi, ⁷⁵Br, ⁷⁶Br, ⁷⁷Br, ^{80m}Br, ¹¹C, ⁵⁵Co, ⁵⁷Co, ⁵⁸Co, ⁵¹Cr, ⁶²Cu, ⁶⁴Cu, ⁶⁷Cu, ¹⁵²Dy, ¹⁶⁶Dy, ¹⁶⁹Er, ¹⁸F, ⁵²Fe, ⁵⁹Fe, ²⁵⁵Fm, ²²¹Fr, ⁶⁷Ga, ⁶⁸Ga, ¹⁵⁴⁻¹⁵⁸Gd, ¹⁰⁷Hg, ¹⁹⁷Hg, ²⁰³Hg, ¹⁶¹Ho, ¹⁶⁶Ho, ¹²⁰I, ¹²³I, ¹²⁴I, ¹²⁵I, ¹²⁶I, ¹³¹I, ¹³³I, ¹¹⁰In, ¹¹¹In, ^{113m}In, ^{114m}In, ¹⁹⁴Ir, ¹⁹²Ir, ¹⁷⁷Lu, ⁵¹Mn, ^{52m}Mn, ⁹⁹Mo, ¹³N, ¹⁵O, ^{189m}Os, ³²P, ³³P, ²¹¹Pb, ²¹²Pb, ¹⁰⁹Pd, ¹⁴⁹Pm, ²¹⁵Po, ¹⁴²Pr, ¹⁴³Pr, ¹⁰⁹Pt, ¹⁹⁷Pt, ²²³Ra, ²²⁶Ra, ^{82m}Rb, ¹⁸⁶Re, ¹⁸⁸Re, 189Re, ¹⁰³Rh, ¹⁰⁵Rh, ²¹⁹Rn, ⁹⁵Ru, ⁹⁷Ru, ^{103m}Ru, ¹⁰⁵Ru, ¹¹⁹Sb, ⁷⁵Se, ¹⁵³Sm, ⁸³Sr, ⁸⁹Sr, ¹⁶¹Tb, ^{94m}Tc, ⁹⁴Tc, ^{99m}Tc, ^{121m}Te, ^{122m}Te, ^{125m}Te, ²⁰¹Tl, ¹⁶⁵Tm, ¹⁶⁷Tm, ¹⁶⁸Tm, ⁸⁶Y, ⁹⁰Y, ¹⁶⁹Yb, ⁸⁹Zr, and a combination thereof.

12. The device according to claim 7, wherein the chemical agent is selected from the group consisting of Cyclohexyl Sarin, GE, Sarin, Soman, Tabun, VE, VG, V-Gas, VM, VX, Distilled Mustard, Lewisite, Nitrogen Mustard, Phosgene Oxime), Ethyldichloroarsine, Lewisite 1, Lewisite 2, Lewisite 3, Methyldichloroarsine, Mustard/Lewisite, Mustard/T, Nitrogen Mustard, Nitrogen Mustard, Phenodichloroarsine, and Sesqui Mustard, Arsine, Cyanogen Chloride, Hydrogen Chloride, Hydrogen Cyanide, Chlorine, Diphosgene, Cyanide, Nitrogen Oxide, Perflurorisobutylene, Phosgene, Red Phosphorous, Sulfur Trioxide-Chlorosulfonic Acid, Teflon and Perflurorisobutylene, Titanium Tetrachloride, Zinc Oxide, Agent 15, BZ, Cannabinoids, Fentanyls, LSD, Phenothiazines, Bromobenzylcyanide, Chloroacetophenone, Chloropicrin, CN in Benzene and Carbon Tetrachloride, CN in Chloroform, CN and Chloropicrin in Chloroform, CR, CS, Adamsite, Diphenylchloroarsine, Diphenylcyanoarsine, opiods, opiates, narcotics, drugs of abuse, and a combination thereof.

13. The device according to claim 7, wherein the biological agent is selected from the group consisting of Anthrax, Cholera, Plague, Botulism, Tularemia, Variola, Q Fever, Staphylococcal Enterotoxin B, Brucellosis, Venezuelan Equine Encephalitis, Tricothecene, Viral hemorrhagic fevers, Filoviruses, Ebola, Marburg, Arenaviruses, Lassa, Machupo, Nipah virus, Melioidosis, Psittacosis, Typhus fever, Viral encephalitis, Alphaviruses, Venezuelan equine encephalitis, Eastern equine encephalitis, Western equine encephalitis, Water safety threats, Hantaviruses, Multidrug-resistant tuberculosis, Nipah virus, Tickborne encephalitis viruses, Tickborne hemorrhagic fever viruses, Yellow fever, and a combination thereof.

14. The device according to claim 1, wherein the contaminant is from protective gear.

15. The device according to claim 14, wherein the protective gear comprises a rubber glove, a rubber apron, a rubber mask, or a rubber boot.

16. The device according to claim 2, wherein the adhesive comprises a polymer.

17. The device according to claim 1, wherein the substrate comprises a polymer.

18. The device according to claim 17, wherein the polymer comprises polyester.

19. The device according to claim 1, wherein the substrate comprises a disc.

20. The device according to claim 19, wherein the disc is about 22-mm in diameter.

21. The device according to claim 1, wherein the housing comprises a polymer.

22. The device according to claim 21, wherein the polymer comprises delron, polyethylene, or polypropylene.

23. The device according to claim 1, wherein the housing comprises a screw mechanism or a stamp mechanism that allows the substrate to protrude beyond the surface of the housing and to be withdrawn into the housing.

24. The device according to claim 1, wherein the cap comprises a snap-on cap or a screw-on cap.

25. The device according to claim 2, further comprising a film that covers the adhesive.

26. A method for collecting a sample from a test surface, wherein the sample comprises a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof comprising:
providing a device comprising a substrate; a housing; and, optionally, a cap, wherein the housing supports the substrate and wherein the cap protects the substrate from contamination;
optionally, advancing the substrate to protrude from the housing;
exposing the substrate;
contacting the substrate with the test surface one or more times;
optionally, withdrawing the substrate into the housing; and
enclosing the substrate.

27. A kit comprising:
a device for collecting a sample from a test surface, comprising a contaminant selected from the group consisting of a dithiocarbamate, a carbamate, a thiuram, disulfiram, a mercaptobenzothiazole and its derivatives, thioureas, 1,3-diphenylguanidine, isothiocyanate, and a combination thereof comprising a substrate; a housing; and, optionally, a cap, wherein the housing supports the substrate and wherein the cap protects the substrate from contamination.
